(19) United States
(12) Patent Application Publication
(10) Pub. No.: US 2026/0285957 A1
(43) Pub. Date: Sep. 24, 2026

(54) ISOFORM-INDEPENDENT ANTIBODIES TO LIPOPROTEIN(A)

(71) Applicant: The Regents of the University of California, Oakland, CA (US)

(72) Inventors: Sotirios Tsimikas, Rancho Santa Fe, CA (US); Joseph L. Witztum, San Diego, CA (US); Ayelet Gonen, La Jolla, CA (US)

(21) Appl. No.: 19/473,760

(22) PCT Filed: Apr. 12, 2024
(86) PCT No.: PCT/US2024/024471

§ 371 Date: Oct. 8, 2025

Related U.S. Application Data
(60) Provisional application No. 63/459,010, filed on Apr. 13, 2023.

(51) Int. Cl. C07K 16/18

(52) U.S. Cl. CPC C07K 16/18; C07K 2317/34; C07K 2317/565; C07K 2317/622; C07K 2319/02

(57) ABSTRACT

The disclosure provides for antibody and antibody fragments that bind to lipoprotein(a) epitopes and methods of use thereof, including the production of transgenic animal models and the use of the fragments as therapeutic and diagnostic agents for treating diseases and disorders associated with Lp(a).

### CROSS REFERENCE TO RELATED APPLICATIONS

This application claims priority to U.S. Provisional Application No. 63/459,010, filed Apr. 13, 2023, the disclosures of which are incorporated herein by reference.

### TECHNICAL FIELD

The disclosure provides human and humanized antibodies, single chain variable region (scFv) and binding domains thereof that bind to lipoprotein(a) (Lp(a)). The disclosure relates to methods and compositions to treat patients with antibodies, scFvs and binding domains thereof to treat diseases and disorders associated with Lp(a) and for use in diagnostics.

### INCORPORATION BY REFERENCE OF SEQUENCE LISTING

Accompanying this filing is a Sequence Listing entitled, “00015-409WO1.xml” created on Apr. 12, 2024 and having 18, 698 bytes of data, machine formatted on IBM-PC, MS-Windows operating system. The sequence listing is hereby incorporated by reference in its entirety for all purposes.

### BACKGROUND

Lipoprotein(a) [Lp(a)] is composed of apolipoprotein(a) covalently bound to apolipoprotein B-100. The apolipoprotein(a) protein displays wide size heterogeneity due to a variable number of kringle IV Type 2 (KIV2) repeats among individuals and populations (see, e.g., FIG. 1A). Apolipoprotein(a) consists of 10 unique kringle IV repeats that are present in one copy, except KIV2 which is present in a variable number of identical copies (1 to >40). It also contains one copy of Kringle V (KV) and an inactive protease-like domain. Plasma Lp(a) levels are genetically determined by the production rate of apolipoprotein(a) in hepatocytes, with isoforms containing a small number of KIV2 repeats being secreted more efficiently, leading to an inverse association of KIV2 repeat number and plasma Lp(a) levels.

Many assays exist to measure plasma Lp(a), but assay methodologies vary among manufacturers and additionally have not been globally standardized. The variability in the number of repeats in KIV2 has led to significant methodological limitations in accurately measuring plasma Lp(a) levels. All commercially available assays use polyclonal antibodies, which are a mixture of antibodies that may bind multiple times to KIV2 repeats and to other segments of apolipoprotein(a). Furthermore, assay calibrators are derived from pooled plasma of multiple donors and generally cannot represent all known apolipoprotein isoforms of the samples being measured. Relative to the calibrators, polyclonal antibodies tend to bind more frequently to larger apolipoprotein(a) isoforms, which are found in subjects with low plasma Lp(a) and thus tend to overestimate these values. In contrast such antibodies bind less frequently to small isoforms, which are found in Lp(a) in subjects with high plasma Lp(a) and tend to underestimate values. This size dependent bias can be minimized by the use of separate pools of calibrators representing most isoforms, as opposed to serial dilutions on one pool, and also be re-assigning new values to the calibrators with a more accurate method.

It is expected that the testing of Lp(a) values will expand with new guidelines recommending every person have an Lp(a) measured at least once in their lifetime and by the development of new therapies substantially lowering Lp(a). Thus the imperative for accurate methods is stronger than ever.

### SUMMARY

The disclosure provides an antibody or antibody fragment that recognizes and binds to lipoprotein(a), wherein the antibody or antibody fragment comprises a variable heavy chain (V_{H}) domain and/or a variable light chain (V_{L}) domain, and wherein (a) the V_{H} domain comprises an amino acid sequence that includes one, two or three complementarity determining regions (CDRs) selected from the group consisting of: SEQ ID NO:9 or variants thereof, SEQ ID NO:10 or variants thereof, and SEQ ID NO:11 or variants thereof; and (b) the V_{L} domain comprises an amino acid sequence that includes one, two or three complementarity determining regions (CDRs) selected from the group consisting of: SEQ ID NO:12 or variants thereof, SEQ ID NO:13 or variants thereof, and SEQ ID NO:14 or variants thereof, wherein the antibody or antibody fragment comprising CDRs selected from SEQ ID Nos:9, 10, 11, 12, 13, and 14, and combinations of any of the foregoing bind to Lp(a). In one embodiment, the V_{H} domain comprises an amino acid sequence that includes CDRs comprising SEQ ID NO:9, 10, and 11. In another embodiment, the V_{L} domain comprises an amino acid sequence that includes CDRs comprising SEQ ID NO:12, 13, and 14. In another or further embodiment, the antibody or antibody fragment is selected from the group consisting of an antibody or scFv with heavy and light chain domains comprising the complementarity determining regions of SEQ ID NO:9, 10, 11, 12, 13, and 14. In yet another or further embodiment, the heavy and light chain domains are linked to an Fc region. In a further embodiment, the Fc region is a human Fc region. In another embodiment, the antibody fragment comprises a single chain variable fragment (“scFv”) that recognizes an epitope of KIV4 of lipoprotein(a). In a further embodiment, the epitope comprises or consists of the sequence FVPPNVI (SEQ ID NO:1). In one embodiment, the epitope contains SEQ ID NO:1 and includes 1-10 additional amino acids at the N-terminal and/or C-terminal end of SEQ ID NO:1. In yet another or further embodiment, the scFv is soluble under physiological conditions. In yet another or further embodiment, the scFv comprises a light-chain variable region having a sequence that is at least 95% identical to the sequence as set forth in SEQ ID NO:16. In still a further embodiment, the scFv comprises a heavy chain variable region having a sequence that is at least 95% identical to the sequence as set forth in SEQ ID NO:18.

The disclosure also provides an antibody comprising a variable light chain and variable heavy chain as described in the prior paragraph. In one embodiment, the antibody is humanized. In another embodiment, the antibody is chimeric.

The disclosure also provides a pharmaceutical composition comprising an antibody or antibody fragment of the disclosure and a pharmaceutically acceptable carrier, excipient, and/or stabilizer.

The disclosure also provide an antibody or antibody fragment of the disclosure bound to a solid substrate.

The disclosure also provides an antibody or antibody fragment of the disclosure operably linked to a detectable label.

The disclosure also provides a polynucleotide that encodes an antibody, antibody fragment, variable light chain, variable heavy chain or scFv of the disclosure. In one embodiment, the polynucleotide comprises SEQ ID NO:15 and/or 17 or variants thereof, wherein the polynucleotide encodes a polypeptide that binds to an epitope of KIV4 of Lp(a) containing SEQ ID NO:1.

The disclosure further includes a vector comprising a polynucleotide encoding an antibody or antibody fragment of the disclosure.

The disclosure also provides a host cell transformed with the polynucleotide of the disclosure.

The disclosure also provides a host cell transformed with a vector of the disclosure.

The disclosure also provides a transgenic animal, comprising a polynucleotide encoding an antibody or antibody fragment of the disclosure. In one embodiment, the animal is a mouse. In another or further embodiment, an scFv or antibody of the disclosure is expressed from hepatocytes and/or macrophages of the transgenic animal. In a further embodiment, the transgenic animal is used to model the effects of coronary artery disease or disorders such as aortic stenosis and/or atherosclerosis.

The disclosure also provides a method of treating or preventing coronary artery disease comprising administering an antibody or antibody fragment or scFv of the disclosure to a subject having or at risk of having coronary artery disease, stroke, peripheral arterial disease or calcific aortic stenosis.

The disclosure also provides a chimeric antigen receptor (CAR) comprising a binding domain of the VH and/or VL domains as described above and herein, wherein the CAR binds to Lp(a).

The disclosure also provides an immunological composition comprising an antigenic peptide comprising the sequence FVPPNVI (SEQ ID NO:1). In one embodiment, the antigenic peptide comprising SEQ ID NO:1 and includes 1-10 additional amino acids at the N-terminal and/or C-terminal end. In one embodiment, a peptide sequence comprising or consisting of SEQ ID NO:1 is linked to or administered with an adjuvant. The disclosure also provides antibodies (polyclonal or monoclonal) obtained from immunizing a mammal with a peptide of SEQ ID NO:1.

The disclosure also provides a CAR-T cell comprising the chimeric antigen receptor of the disclosure.

### DESCRIPTION OF DRAWINGS

FIG. 1A-E shows (A) a cartoon of apolipoprotein(a) and KIV domains. (B)-(E) show a cartoon of the methodology of Lp(a) and total apo(a) assays. Panels B and C represent Lp(a) and total apo(a) assays, using capture antibodies MB47 and LPA4, respectively, and detecting antibody biotin-LPA4. Panels D and E represent Lp(a) and total apo(a) assays, using capture antibodies MB47 and LPA4, respectively, and detecting antibody biotin-LPA-KIV4.

FIG. 2 shows IgG titers against K8 in immunized mice plasma.

FIG. 3 shows representative 8 colonies screened against antigens arrays. Colony #6 was subcloned in semisolid-gel to ensure monoclonality.

FIG. 4 shows that the subcolony 1F4 was cultured and the Ab was purified by BioXCell and named LPA-KIV4.

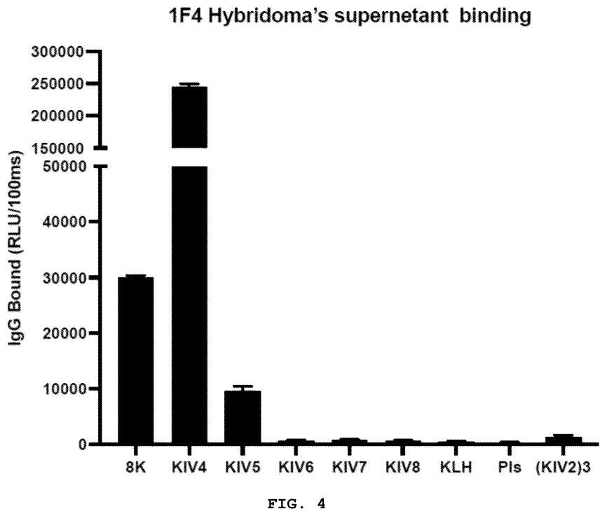

FIG. 5 shows a dot-blot of reactivity of the Ab from hybridoma 1F4 against 8K, peptide KIV4, Peptide KIV5, plasminogen and KIV2(3).

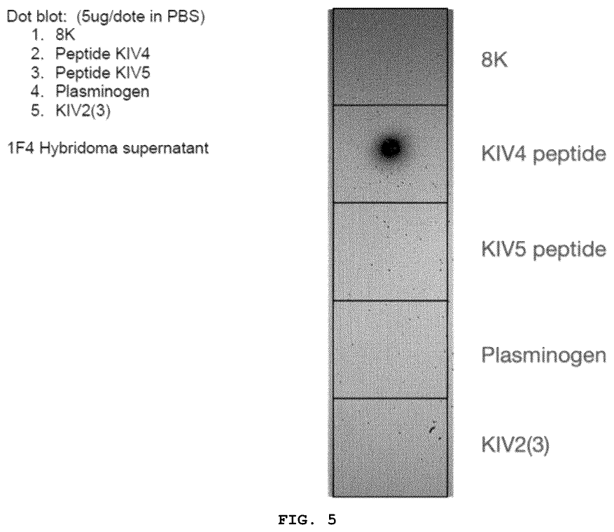

### DETAILED DESCRIPTION

As used herein and in the appended claims, the singular forms “a,” “an,” and “the” include plural referents unless the context clearly dictates otherwise. Thus, for example, reference to a “single-chain variable fragment” or “scFv” includes a plurality of single-chain variable fragments and reference to “lipoprotein(a)” includes reference to one or more lipoprotein(a) molecules and equivalents thereof known to those skilled in the art, and so forth.

All publications mentioned herein are incorporated herein by reference in full for the purpose of describing and disclosing the methodologies, which are described in the publications, which might be used in connection with the description herein. The publications discussed above and throughout the text are provided solely for their disclosure prior to the filing date of the present application. Nothing herein is to be construed as an admission that the inventors are not entitled to antedate such disclosure by virtue of prior disclosure. Moreover, with respect to any term that is presented in one or more publications that is similar to, or identical with, a term that has been expressly defined in this disclosure, the definition of the term as expressly provided in this disclosure will control in all respects.

Also, the use of “and” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

Unless defined otherwise, all technical and scientific terms used herein have the same meaning as commonly understood to one of ordinary skill in the art to which this disclosure belongs. Although any methods and reagents similar or equivalent to those described herein can be used in the practice of the disclosed methods and compositions, the exemplary methods and materials are now described.

Lipoprotein(a) (Lp(a)) is common in the human population. There is currently no standardized method or compositions for measuring Lp(a) content in a biological sample. Current antibodies being used to measure Lp(a) bind to kringle repeats (type-2) and thus depending upon how many repeats are present result in obscure measurements.

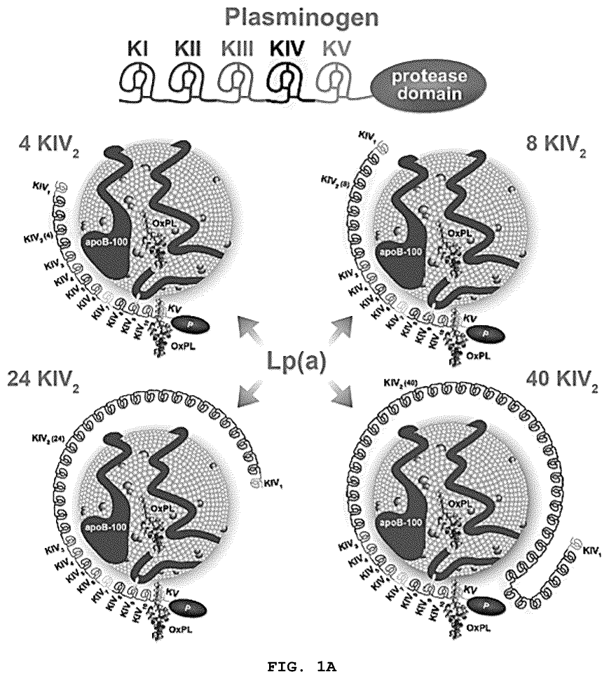

“Lipoprotein(a)” or “Lp(a)” refer to a low-density lipoprotein variant containing a protein called apolipoprotein(a) (Apo (a)). Genetic and epidemiological studies have identified lipoprotein(a) as a risk factor for atherosclerosis and related diseases, such as coronary heart disease and stroke. Lipoprotein(a) consists of an LDL-like particle and the specific apolipoprotein(a), which is bound covalently to the apoB contained in the outer shell of the particle. Apo (a) proteins vary in size due to a size polymorphism [KIV-2 VNTR], which is caused by a variable number of kringle IV repeats in the LPA gene. This size variation at the gene level is expressed on the protein level as well, resulting in apo(a) proteins with 10 to more than 50 kringle IV repeats (each of the variable kringle IV domains consists of 114 amino acids). These variable apo(a) sizes are known as “apo(a) isoforms”. There is a general inverse correlation between the size of the apo(a) isoform and the Lp(a) plasma concentration. Lp(a) concentrations can vary by more than one thousand between individuals, from <0.2 to >200 mg/dl. There is a two- to three-fold higher mean Lp(a) plasma concentration in populations of African descent compared to Asian, Oceanic, or European populations. The general inverse correlation between apo(a) isoform size and Lp(a) plasma concentration is observed in all populations. Cartoon depictions of Lp(a) are provided in FIG. 1A.

Lp(a) contributes to the process of atherogenesis. The structure of apolipoprotein(a) is similar to plasminogen and tPA (tissue plasminogen activator) and it competes with plasminogen for its binding site, leading to reduced fibrinolysis. Also, because Lp(a) stimulates secretion of PAI-1, it leads to thrombogenesis. It also may enhance coagulation by inhibiting the function of tissue factor pathway inhibitor. Moreover, Lp(a) carries atherosclerosis-causing cholesterol and binds atherogenic pro-inflammatory oxidized phospholipids as a preferential carrier of oxidized phospholipids in human plasma, which attracts inflammatory cells to vessel walls and leads to smooth muscle cell proliferation. Lp(a) also is hypothesized to be involved in wound healing and tissue repair by interacting with components of the vascular wall and extracellular matrix. Apo (a), a distinct feature of the Lp(a) particle, binds to immobilized fibronectin thereby providing Lp(a) with the serine-proteinase-type proteolytic activity.

High Lp(a) in blood correlates with coronary heart disease (CHD), cardiovascular disease (CVD), atherosclerosis, thrombosis, and stroke. High Lp(a) correlates with early atherosclerosis independently of other cardiac risk factors, including LDL. In patients with advanced cardiovascular disease, Lp(a) indicates a coagulant risk of plaque thrombosis. Apo (a) contains domains that are very similar to plasminogen (PLG). Lp(a) accumulates in the vessel wall and inhibits the binding of PLG to the cell surface, reducing plasmin generation, which increases clotting. This inhibition of PLG by Lp(a) also promotes the proliferation of smooth muscle cells. These unique features of Lp(a) suggest that Lp(a) causes generation of clots and atherosclerosis. Numerous studies confirming a strong correlation between elevated Lp(a) and heart disease have led to the consensus that Lp(a) is an important independent predictor of cardiovascular disease. Animal studies have shown that Lp(a) may directly contribute to atherosclerotic damage by increasing plaque size, inflammation, instability, and smooth muscle cell growth. Genetic data also support the theory that Lp(a) causes cardiovascular disease. Lp(a) is associated with enhanced atherogenic potential, particularly at levels >30 mg/dl, and is shown to be an independent predictor (odds ratio −1.5-2) of cardiovascular risk, particularly in younger subjects (<60 years old) and those with elevated LDL cholesterol levels.

Lp(a) appears with different isoforms (per kringle repeats) of apolipoprotein; 40% of the variation in Lp(a) levels when measured in mg/dl can be attributed to different isoforms. Lighter Lp(a) are also associated with disease. Thus, a test with simple quantitative results may not provide a complete assessment of risk.

As described above and in additional detail in the Examples, the disclosure provides a novel monoclonal antibody, LPA-KIV4, that detects a unique 7-amino acid epitope on KIV4 of apolipoprotein(a) present only in one copy. LPA-KIV4 successfully quantitated a wide range of plasma Lp(a) levels in a chemiluminescent ELISA format and demonstrated staining of human carotid endarterectomy specimens. This antibody can be used in research settings to immunologically detect apolipoprotein(a) and in clinical settings to measure plasma Lp(a) levels. Since it is a monoclonal antibody binding only one unique epitope of apolipoprotein(a), it may overcome many of the current limitations in measuring Lp(a) levels.

Increasing evidence suggests that Lp(a) is an independent, genetic and likely causal risk factor for cardiovascular disease (CVD). Elevated Lp(a) confers higher risk for myocardial infarction, stroke, peripheral arterial disease and calcific aortic valve stenosis. The association of Lp(a) with risk of CVD is approximately linear, reaching 2-4 fold higher risk at the highest Lp(a) levels. Lp(a) levels may vary more than 1000-fold between individuals, from <0.1 to >300 mg/dl (>750 nmol/L).

Elevated Lp(a) is highly prevalent, with an estimated 20% of the population having levels >50 mg/dL (>125 nmol/L), the threshold above which risk accrues in statin-treated patients. Furthermore, it is a target of therapy to reduce risk of cardiovascular disease, with recent studies showing that antisense oligonucleotides may lower Lp(a) by over 80%, paving the way for a phase 3 clinical trial that is now underway (Lp(a) HORIZON, Assessing the Impact of Lipoprotein(a) Lowering With TQJ230 on Major Cardiovascular Events in Patients With CVD, [https://]clinicaltrials.gov/ct2/show/NCT04023552). Furthermore, multiple national and international societies have suggested that Lp(a) be measured in individuals at moderate to high risk for CVD to enhance risk prediction or for considerations as a risk enhancer. The EAS/ESC has also recommended Lp(a) be measured at least once in everyone's lifetime to assess cardiovascular risk, creating a strong imperative to develop more accurate methods to measure Lp(a) in the clinic.

In 2018, an NHLBI Working Group recommended the development of a globally-standardized measurement of Lp(a) applicable to commercial laboratories. The current methods of measuring Lp(a) are either to 1): assign target values to the assay calibrators in terms of total Lp(a) mass [apo(a), apoB, lipid and carbohydrate components] and represent values in mg/dL. These methods however are limited in having no traceability of the various calibrators to any established reference material; or 2): assign target values to assay calibrators traceable to the World Health Organization/International Federation of Clinical Chemistry and Laboratory Medicine secondary reference material PRM-2B and representing values in nmol/L (i.e. molar concentration of Lp(a) particles). The NHLBI group also recommended that reporting total Lp(a) mass in mg/dL be discontinued and instead that values should be reported in nmol/L traceable to a common reference system. Furthermore, harmonization of results obtained by the different methods should be performed after an accuracy-based common calibration is performed.

The lack of widely-available monoclonal antibodies to Lp(a) that do not bind to either KIV2 repeats or cross-react with plasminogen, which has substantial homology to apolipoprotein(a), has hindered both the accurate measurement of Lp(a) levels as well as the global standardization of Lp(a) assays. The difficulty in generating monoclonal antibodies to Lp(a) suggests that only a limited number of antigenic sites are available to generate such antibodies. One such monoclonal antibody described is a40 which binds to an unknown site on KIV9 and has been used in an ELISA format to measure Lp(a). An ELISA format using a capture antibody detecting KIV2 and a second detection antibody that only binds to one unique site on apolipoprotein(a), along with calibrators traced to the WHO/IFCC, is thought to be the optimal method to reducing size dependent bias. The disclosure provides methods and compositions to carry out and such an ELISA and methods.

Since commercially available assays generally use polyclonal antibodies, these antibodies are isoform-dependent by virtue of binding multiple times to the identical KIV2 repeat segment. In some assays, bias to apolipoprotein(a) size can be somewhat minimized by the use of independent calibrators each containing a suitable distribution of apo(a) isoforms and that have been accurately assigned values by a separate isoform independent assay. Despite this, most assays remain sensitive to apolipoprotein(a) size due to a lack of a consistent relationship between Lp(a) levels and apo(a) isoform size. For this reason, high or low Lp(a) levels may be observed in samples encompassing a relatively large range of apolipoprotein(a) size. Reflecting the size heterogeneity bias, a recent study evaluating Lp(a) levels by 6 commercially available methods concluded that current commercial immunological assays are differently-calibrated and their biases differed significantly across the clinically-relevant concentration range in a non-linear manner not entirely dependent on apolipoprotein(a) phenotypes. Based on these findings, it was suggested that global harmonization of methods is required with further improvement in methodological techniques.

The disclosure demonstrates that LPA-KIV4 can be used successfully to measure Lp(a) levels and compares favorably to previously validated in-house research assays. The current data also shows a significant correlation between “true Lp(a)” versus “total apo(a)” assays. Since in most setting there is a minimal amount of circulating free apolipoprotein(a), this type of distinction is not usually needed. However, in the current PCSK9 inhibitor era it is possible that these assays may provide a method to ascertain the nature of circulating Lp(a)/apolipoprotein(a) particles as suggested. The LPA-KIV4 can be used alone or in combination with an LPA-KIV9 antibody.

The disclosure provides compositions and methods that measures Lp(a) by binding to non-repeat epitopes thereby eliminating inaccuracies and creating a standardizable method for measuring Lp(a). The disclosure provides antibody and antibody fragments that only bind to non-repeats and thus provides a more accurate measurement of Lp(a) content.

The term “about” when referring to a measurable value such as an amount, a temporal duration, and the like, is meant to encompass variations of ±20% or in some instances ±10%, or in some instances ±5%, or in some instances ±1%, or in some instances ±0.1% from the specified value, as such variations are appropriate to perform the disclosed methods or describe the compositions herein. Moreover, any value or range (e.g., less than 20 or similar terminology) explicitly includes any integer between such values or up to the value. Thus, for example, “one to five mutations” explicitly includes 1, 2, 3, 4, and/or 5 mutations.

The terms “antibody” and “immunoglobulin” are used interchangeably in the broadest sense and include monoclonal antibodies (e.g., full length or intact monoclonal antibodies), polyclonal antibodies, multivalent antibodies, multispecific antibodies (e.g., bispecific antibodies so long as they exhibit the desired biological activity) and also includes antibody fragments. An antibody can be human, humanized and/or affinity matured.

Depending on the amino acid sequence of the constant domain of the antibody heavy chains, immunoglobulins can be assigned to different classes. There are five major classes of immunoglobulins: IgA, IgD, IgE, IgG, and IgM, and several of these can be further divided into subclasses (isotypes), e.g., IgG₁, IgG₂, IgG₃, IgG₄, IgA₁, and IgA₂. The heavy-chain constant domains that correspond to the different classes of immunoglobulins are called α, δ, ε, γ, and μ, respectively. The subunit structures and three-dimensional configurations of different classes of immunoglobulins are known.

“Antibody fragments” comprise only a portion of an intact antibody, wherein the portion typically retains at least one, more commonly most, or all, of the functions normally associated with that portion of the antibody when present in an intact antibody. Examples of antibody fragments include Fab, Fab′, F(ab′)₂, and Fv fragments; diabodies; linear antibodies; single-chain antibody molecules (scFv); and multispecific antibodies formed from antibody fragments. In one embodiment, an antibody fragment comprises an antigen binding site of the intact antibody and thus retains the ability to bind its cognate antigen. Typically such an antibody fragment will have all of the CDRs. In another embodiment, an antibody fragment, for example one that comprises the Fc region, retains at least one of the biological functions normally associated with the Fc region when present in an intact antibody, such as FcR binding, antibody half-life modulation, ADCC function and complement binding. In one embodiment, an antibody fragment is a monovalent antibody that has an in vivo half-life substantially similar to an intact antibody. For example, such an antibody fragment may comprise an antigen binding arm linked to an Fc sequence capable of conferring in vivo stability to the fragment.

The term “antibody heavy chain,” refers to the larger of the two types of polypeptide chains present in antibody molecules in their naturally occurring conformations, and which normally determines the class to which the antibody belongs.

The term “antibody light chain,” refers to the smaller of the two types of polypeptide chains present in antibody molecules in their naturally occurring conformations. Kappa (κ) and lambda (λ) light chains refer to the two major antibody light chain isotypes.

An “antigen” is a target to which an antibody can selectively bind. The target antigen may be polypeptide, carbohydrate, nucleic acid, lipid, hapten, small molecule, or other naturally occurring or synthetic compound. In one embodiment of the disclosure an antigen is Lp(a). In another embodiment, the antibody binds to an epitope on the antigen comprising LVPPNVI (SEQ ID NO:1), CSDAEWTAFVPPNVILAPSLEAFFEQ (SEQ ID NO:2).

The term “array,” as used herein, generally refers to a predetermined spatial arrangement of binding islands, biomolecules, or spatial arrangements of binding islands or biomolecules. Arrays according to the disclosure that include biomolecules immobilized on a surface may also be referred to as “biomolecule arrays.” Arrays according to the disclosure that comprise surfaces that are activated, adapted, prepared, or modified to facilitate the binding of biomolecules to the surface may also be referred to as “binding arrays.” Further, the term “array” may be used herein to refer to multiple arrays arranged on a surface, such as would be the case where a surface bore multiple copies of an array. Such surfaces bearing multiple arrays may also be referred to as “multiple arrays” or “repeating arrays.” The use of the term “array” herein may encompass biomolecule arrays, binding arrays, multiple arrays, and any combination thereof; the appropriate meaning will be apparent from context. The biological sample can include fluid or solid samples from any tissue of the body including plasma.

An array of the disclosure comprises a substrate. By “substrate” or “solid support” or other grammatical equivalents, herein is meant any material appropriate for the attachment of biomolecules and is amenable to at least one detection method. As will be appreciated by those in the art, the number of possible substrates is very large. Possible substrates include, but are not limited to, glass and modified or functionalized glass, plastics (including acrylics, polystyrene and copolymers of styrene and other materials, polypropylene, polyethylene, polybutylene, polyurethanes, TEFLON, etc.), polysaccharides, nylon or nitrocellulose, resins, silica or silica-based materials including silicon and modified silicon, carbon, metals, inorganic glasses, plastics, ceramics, and a variety of other polymers. In addition, as is known the art, the substrate may be coated with any number of materials, including polymers, such as dextrans, acrylamides, gelatins or agarose. Such coatings can facilitate the use of the array with a biological sample derived from serum. In one embodiment, the biomolecule is an antibody or antibody fragment or derivative of the disclosure.

A planar array of the disclosure will generally contain addressable locations (e.g., “pads”, “addresses,” or “micro-locations”) of biomolecules in an array format. The size of the array will depend on the composition and end use of the array. Arrays containing from about 2 different biomolecules to many thousands can be made. In some embodiments, the compositions of the disclosure may not be in an array format; that is, for some embodiments, compositions comprising a single biomolecule may be made as well. In addition, in some arrays, multiple substrates may be used, either of different or identical compositions. Thus, for example, large planar arrays may comprise a plurality of smaller substrates. In one embodiment, the biomolecule is an antibody or antibody fragment or derivative of the disclosure.

As an alternative to planar arrays, bead based assays in combination with flow cytometry have been developed to perform multiparametric immunoassays. In bead based assay systems the biomolecules can be immobilized on addressable microspheres. Each biomolecule for each individual immunoassay is coupled to a distinct type of microsphere (i.e., “microbead”) and the immunoassay reaction takes place on the surface of the microspheres. Dyed microspheres with discrete fluorescence intensities are loaded separately with their appropriate biomolecules. The different bead sets carrying different capture probes can be pooled as necessary to generate custom bead arrays. Bead arrays are then incubated with the sample in a single reaction vessel to perform the immunoassay. In one embodiment, the biomolecule is an antibody or antibody fragment or derivative of the disclosure.

The term “anti-Lp(a) antibody” or “an antibody that binds to Lp(a)” refers to an antibody that is capable of binding Lp(a) with sufficient affinity such that the antibody is useful as a diagnostic and/or therapeutic agent in targeting Lp(a). In some embodiments of the disclosure an anti-Lp(a) antibody binds specifically to KIV₄ without binding to KIV₉ or KIV₂ or plasminogen. In a specific embodiment, the antibody or antibody fragment binds to an epitope containing or comprising LVPPNVI (SEQ ID NO:1).

As used herein “LPA-KIV₉ antibody” refers to the monoclonal antibody and fragments thereof disclosed in PCT/US2021/029307 (WO/2021/222181), the disclosures of which are incorporated herein by reference for all purposes. In particular, an LPA-KIV₉ antibody binds to an epitope comprising or consisting of LETPTVV (SEQ ID NO:7) or CSETESGVLETPTVVPVPSMEAH (SEQ ID NO:8).

A “blocking” antibody or an “antagonist” antibody is one which inhibits or reduces biological activity of the antigen it binds. Certain blocking antibodies or antagonist antibodies substantially or completely inhibit the biological activity of the antigen. In one embodiment, a blocking antibody binds to its antigen but does not bind an FcR or cause any ADCC. Thus, in one embodiment, the antibody or antibody fragment binds its antigen and prevents or inhibits the antigen's biological activity while the antibody or antibody fragment itself does not induce or has limited effect on the immune system.

“Binding affinity” generally refers to the strength of the sum total of non-covalent interactions between a binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule ‘X’ for its partner ‘Y’ can generally be represented by the dissociation constant (K_{d}). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the disclosure.

“Binds the same epitope as” means the ability of an antibody, scFv, or other antigen binding domain to bind to a target antigen having the same epitope as the exemplified antibody, scFv, or other antigen binding domain. As an example, the epitopes of the exemplified antibody, scFv, or other binding agent and other antibodies can be determined using standard epitope mapping techniques. Epitope mapping techniques, well known in the art include Epitope Mapping Protocols in Methods in Molecular Biology, Vol. 66 (Glenn E. Morris, Ed., 1996) Humana Press, Totowa, New Jersey. For example, linear epitopes may be determined by, e.g., concurrently synthesizing large numbers of peptides on solid supports, the peptides corresponding to portions of the protein molecule, and reacting the peptides with antibodies while the peptides are still attached to the supports. Such techniques are known in the art and described in, e.g., U.S. Pat. No. 4,708,871; Geysen et al., (1984) Proc. Natl. Acad. Sci. USA 8:3998-4002; Geysen et al., (1985) Proc. Natl. Acad. Sci. USA 82:78-182; Geysen et al., (1986) Mol. Immunol. 23:709-715. The epitope bound by the antibody or antibody fragment of the disclosure can be determined by an Epitope Binning assay. Epitope binning is a competitive immunoassay used to characterize and then sort a library of monoclonal antibodies against a target protein. Antibodies against a similar target are tested against all other antibodies in the library in a pairwise fashion to see if antibodies block one another's binding to the epitope of an antigen. After each antibody has a profile created against all of the other antibodies in the library, a competitive blocking profile is created for each antibody relative to the others in the library. Closely related binning profiles indicate that the antibodies have the same or a closely related epitope and are “binned” together. Similarly, conformational epitopes are readily identified by determining spatial conformation of amino acids such as by, e.g., hydrogen/deuterium exchange, x-ray crystallography and two-dimensional nuclear magnetic resonance. Antigenic regions of proteins can also be identified using standard antigenicity and hydropathy plots, such as those calculated using, e.g., the Omiga version 1.0 software program available from the Oxford Molecular Group. This computer program employs the Hopp/Woods method, Hopp et al, (1981) Proc. Natl. Acad. Sci USA 78:3824-3828; for determining antigenicity profiles, and the Kyte-Doolittle technique, Kyte et al, (1982) J. Mol. Biol. 157:105-132; for hydropathy plots. To determine if selected monoclonal antibodies against a target (e.g., Lp(a)) bind to unique epitopes, each antibody can be biotinylated using commercially available reagents. Competition studies using unlabeled monoclonal antibodies and biotinylated monoclonal antibodies can be performed using Lp(a)-coated-ELISA plates. Biotinylated mAb binding can be detected with a strep-avidin-alkaline phosphatase probe.

A “biological sample” encompasses a variety of sample types obtained from an individual and can be used in a diagnostic or monitoring assay. The definition encompasses blood, plasma, serum, sputum, cerebral spinal fluid, urine and other liquid samples of biological origin; solid tissue samples such as a biopsy specimen; or tissue cultures or cells derived therefrom, and the progeny thereof. The definition also includes samples that have been manipulated in any way after their procurement, such as by treatment with reagents, solubilization, or enrichment for certain components, such as proteins or polynucleotides, or embedding in a semi-solid or solid matrix for sectioning purposes. The source of the biological sample may be solid tissue as from a fresh, frozen and/or preserved organ or tissue sample or biopsy or aspirate; blood or any blood constituents; bodily fluids such as cerebral spinal fluid, amniotic fluid, peritoneal fluid, or interstitial fluid; cells from any time in gestation or development of the subject. In some embodiments, the biological sample is obtained from a site of inflammation, site of a tumor or site of coronary or vascular disease. The biological sample may contain compounds which are not naturally intermixed with the tissue in nature such as preservatives, anticoagulants, buffers, fixatives, nutrients, antibiotics, or the like.

A “conservative substitution” or “conservative sequence modifications” refers to amino acid modifications that do not significantly affect or alter the binding characteristics or function of a protein such as an antibody or antibody fragment. For example, “conservative sequence modifications” refers to amino acid modifications that do not significantly affect or alter the binding characteristics or function of the antibody, antibody fragment, or non-immunoglobulin binding domains. Such conservative modifications include amino acid substitutions, additions and deletions. Modifications can be introduced into an antibody or antibody fragment, the non-immunoglobulin binding domain or other proteins or polypeptides of the disclosure by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within an anti-Lp(a) antibody or fragment of the disclosure can be replaced with other amino acid residues from the same side chain family and the altered antibody or antibody fragment can be tested using the binding and/or functional assays described herein.

The term “diabodies” refers to small antibody fragments with two antigen-binding sites, which fragments comprise a heavy-chain variable domain (V_{H}) connected to a light-chain variable domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90:6444-6448 (1993). Triabodies and tetrabodies are also described in Hudson et al., Nat. Med. 9:129-134 (2003).

A “disorder” or “disease” is any condition that would benefit from treatment or diagnosis with a substance/molecule or method of the disclosure. This includes chronic and acute disorders or diseases including those pathological conditions which predispose the mammal to the disorder in question. Non-limiting examples of disorders to be treated herein include cardiovascular disease, stenosis, liver disease (e.g., NASH or NALFD), Kawasaki disease, aging-associated diseases and disorders, senescence or inflammatory diseases attributable to Lp(a).

“Derived from” as that term is used herein, indicates a relationship between a first and a second molecule. It generally refers to structural similarity between the first molecule and a second molecule and does not connotate or include a process or source limitation on a first molecule that is derived from a second molecule. For example, in the case of an antibody fragment that is derived from an antibody molecule, the antibody fragment retains sufficient antibody structure such that is has the required function, namely, the ability to bind to an antigen. It does not connotate or include a limitation to a particular process of producing the antibody fragment.

Antibody “effector functions” refer to those biological activities attributable to the Fc region of an antibody, and vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g. B cell receptor); and B cell activation.

As used herein, an “epitope” is defined to be the portion of an antigen capable of eliciting an immune response, or the portion of an antigen that binds to an antibody or antibody fragment. Epitopes can be a protein sequence or subsequence. In one embodiment, an epitope of the disclosure is a peptide that comprises or consists of SEQ ID NO:1.

The term “expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, including cosmids, plasmids (e.g., naked or contained in liposomes) and viruses (e.g., lentiviruses, retroviruses, adenoviruses, and adeno-associated viruses) that incorporate the recombinant polynucleotide.

“Fc receptor” or “FcR” describes a receptor that binds to the Fc region of an antibody. In some embodiments, an FcR is a native human FcR. In some embodiments, an FcR is one which binds an IgG antibody (a gamma receptor) and includes receptors of the FCγRI, FCγRII, and FcγRIII subclasses, including allelic variants and alternatively spliced forms of those receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain. (see, e.g., Daeron, Annu. Rev. Immunol. 15:203-234 (1997)). FcRs are reviewed, for example, in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein.

Fc receptor also include the neonatal receptor, FcRn, which is responsible for the transfer of maternal IgGs to the fetus (Guyer et al., J. Immunol. 117:587 (1976) and Kim et al., J. Immunol. 24:249 (1994)) and regulation of homeostasis of immunoglobulins. Methods of measuring binding to FcRn are known (see, e.g., Ghetie and Ward., Immunol. Today 18 (12): 592-598 (1997); Ghetie et al., Nature Biotechnology, 15 (7): 637-640 (1997); Hinton et al., J. Biol. Chem. 279 (8): 6213-6216 (2004); WO 2004/92219 (Hinton et al.).

Binding to human FcRn in vivo and serum half-life of human FcRn high affinity binding polypeptides can be assayed, e.g., in transgenic mice or transfected human cell lines expressing human FcRn, or in primates to which the polypeptides with a variant Fc region are administered. WO 2000/42072 (Presta) describes antibody variants with improved or diminished binding to FcRs. See also, e.g., Shields et al. J. Biol. Chem. 9 (2): 6591-6604 (2001), each of which is herein incorporated by reference in its entirety.

The term “Fc region” as used herein refers to the C-terminal region of an immunoglobulin heavy chain, including native sequence Fc regions and variant Fc regions.

A “functional Fc region” possesses an “effector function” of a native sequence Fc region. Such effector functions generally require the Fc region to be combined with a binding to domain (e.g., an antibody variable domain) and can be assessed using various assays (e.g., as described in references cited herein).

A “native sequence Fc region” comprises an amino acid sequence that is identical to the amino acid sequence of an Fc region found in nature. Native sequence human Fc regions include a native sequence human IgG1 Fc region (non-A and A allotypes); native sequence human IgG2 Fc region; native sequence human IgG3 Fc region; and native sequence human IgG4 Fc region as well as naturally occurring variants thereof.

“Fv” is the minimum antibody fragment which contains a complete antigen-recognition and antigen-binding site. In a two-chain Fv species, this region is composed of a dimer of one heavy- and one light-chain variable domain in tight, non-covalent association. In a single-chain Fv (scFv) species, one heavy- and one light-chain variable domain can be covalently linked by a flexible peptide linker such that the light and heavy chains can associate in a “dimeric” structure analogous to that in a two-chain Fv species. It is in this configuration that the three hypervariable regions (HVRs; sometimes referred to a CDRs) of each variable domain interact to define an antigen-binding site on the surface of the V_{H}-V_{L} dimer. Collectively, the six HVRs (or CDRs) confer antigen-binding specificity to the antibody. However, even a single variable domain (or half of an Fv comprising only three HVRs specific for an antigen) has the ability to recognize and bind antigen, although at a lower affinity than the entire binding site.

A “Fab fragment” refers to an antibody fragment that contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains have a free thiol group. F(ab′)₂ antibody fragments originally were produced as pairs of Fab′ fragments which have hinge cysteines between them. Other chemical couplings of antibody fragments are also known.

Papain digestion of antibodies produces two identical antigen-binding Fab fragments, each with a single antigen-binding site, and a residual “Fc” fragment, whose name reflects its ability to crystallize readily. Pepsin treatment yields an F(ab′)₂ fragment that has two antigen-combining sites and is still capable of cross-linking antigen.

“Framework” or “FR” residues are those variable domain residues other than the HVR (or CDR) residues as herein defined. Framework residues typically have variability without effecting the binding specificity of the antibody. Conservative amino acid substitutions can be readily performed in the framework regions of the antibody without effecting the bindings specificity of the antibody. Accordingly, the disclosure encompasses antibody or antibody fragments wherein the framework regions can vary from a reference sequence by 80%, 90%, 95%, 98%, or 99% identity so long as the CDRs are not substantially varied or so long as the CDRs are not varied.

“Humanized” forms of non-human (e.g., murine) antibodies are chimeric antibodies that contain minimal sequence derived from non-human immunoglobulin. In one embodiment, a humanized antibody is a human immunoglobulin (recipient antibody) in which residues from a HVR (CDR) of the recipient are replaced by residues from a HVR (CDR) of a non-human species (donor antibody) such as mouse, rat, rabbit, or nonhuman primate having the desired specificity, affinity, and/or capacity. In some instances, FR residues of the human immunoglobulin are replaced by corresponding non-human residues. Furthermore, humanized antibodies may comprise residues that are not found in the recipient antibody or in the donor antibody. These modifications may be made to further refine antibody performance. In general, a humanized antibody comprises substantially all of at least one, and typically two, variable domains, in which all or substantially all of the hypervariable loops correspond to those of a non-human immunoglobulin, and all or substantially all of the FRs are those of a human immunoglobulin sequence. The humanized antibody optionally also comprise at least a portion of an immunoglobulin constant region (Fc), typically that of a human immunoglobulin. For further details, see, e.g., Jones et al., Nature 321:522-525 (1986); Riechmann et al., Nature 332:323-329 (1988); and Presta, Curr. Op. Struct. Biol. 2:593-596 (1992). See also, e.g., Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994); and U.S. Pat. Nos. 6,982,321 and 7,087,409.

A “human antibody” is an antibody that possesses an amino acid sequence which corresponds to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries. Hoogenboom and Winter, J. Mol. Biol., 227:381 (1991); Marks et al., J. Mol. Biol., 222:581 (1991), herein incorporated by reference in its entirety. Also available for the preparation of human monoclonal antibodies are methods described in Cole et al., Monoclonal Antibodies and Cancer Therapy, Alan R. Liss, p. 77 (1985); Boerner et al., J. Immunol., 147 (1): 86-95 (1991). See also van Dijk and van de Winkel, Curr. Opin. Pharmacol., 5:368-74 (2001), each of which is herein incorporated by reference in its entirety. Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584). See also, for example, Li et al., Proc. Natl. Acad. Sci. USA, 103:3557-3562 (2006) regarding human antibodies generated via a human B-cell hybridoma technology, each of which is herein incorporated by reference in its entirety.

“Human effector cells” are leukocytes which express one or more FcRs and perform effector functions. In certain embodiments, the cells express at least FcγRIII and perform ADCC effector function(s). Examples of human leukocytes which mediate ADCC include peripheral blood mononuclear cells (PBMC), natural killer (NK) cells, monocytes, cytotoxic T cells, and neutrophils. The effector cells may be isolated from a native source, e.g., from blood.

The term “hypervariable region,” “HVR,” or “HV,” (sometimes referred to as complementarity-determining regions (“CDR”)) when used herein refers to the regions of an antibody variable domain which are hypervariable in sequence and/or form structurally defined loops. Generally, antibodies comprise six HVRs (CDRs); three in the V_{H} (H1, H2, H3), and three in the V_{L} (L1, L2, L3). In native antibodies, H3 and L3 display the most diversity of the six HVRs, and H3 in particular is believed to play a unique role in conferring fine specificity to antibodies. See, e.g., Xu et al., Immunity 13:37-45 (2000); Johnson and Wu, in Methods in Molecular Biology 248:1-25 (Lo, ed., Human Press, Totowa, N.J., 2003). Indeed, naturally occurring camelid antibodies consisting of a heavy chain only are functional and stable in the absence of light chain. See, e.g., Hamers-Casterman et al., Nature 363:446-448 (1993); Sheriff et al., Nature Struct. Biol. 3:733-736 (1996).

An “isolated” antibody, antibody fragment and the like refer to an antibody, antibody fragment or derivative, which has been identified and separated and/or recovered from a component of its natural environment. Contaminant components of its natural environment include materials which would interfere with diagnostic or therapeutic uses for the antibody or antibody fragment, and may include enzymes, hormones, lipids, and other proteinaceous or nonproteinaceous solutes. In some embodiments, the antibody or antibody fragment is purified (1) to greater than 95% by weight as determined by the Lowry method, and typically more than 99% by weight, (2) to a degree sufficient to obtain at least 15 residues of N-terminal or internal amino acid sequence by use of a spinning cup sequenator, and/or (3) to homogeneity by SDS-PAGE under reducing or nonreducing conditions using Coomassie blue or silver stain. An isolated antibody or antibody fragment includes the antibody or antibody fragment in situ within recombinant cells since at least one component of the antibody's natural environment is not present. Ordinarily, however, an isolated antibody or antibody fragment is prepared by at least one purification step.

An “isolated” nucleic acid molecule is a nucleic acid molecule that is identified and separated from at least one contaminant nucleic acid molecule with which it is ordinarily associated in the natural source of the antibody nucleic acid. An isolated nucleic acid molecule is other than in the form or setting in which it is found in nature. Isolated nucleic acid molecules therefore are distinguished from the nucleic acid molecule as it exists in natural cells. However, an isolated nucleic acid molecule includes a nucleic acid molecule contained in cells that ordinarily express the antibody where, for example, the nucleic acid molecule is in a chromosomal location different from that of natural cells.

The term “label” when used herein refers to a compound or composition which is conjugated or fused directly or indirectly to a reagent such as a nucleic acid probe or an antibody or antibody fragment and facilitates detection of the reagent to which it is conjugated or fused. The label may itself be detectable (e.g., radioisotope labels, luminescent or fluorescent labels) or, in the case of an enzymatic label, may catalyze chemical alteration of a substrate compound or composition which is detectable.

The term “monoclonal antibody” as used herein refers to an antibody obtained from a population of substantially homogeneous antibodies, i.e., the individual antibodies comprising the population are identical except for possible mutations, e.g., naturally occurring mutations, that may be present in minor amounts. Thus, the modifier term “monoclonal” indicates the character of the antibody as not being a mixture of discrete antibodies. In certain embodiments, such a monoclonal antibody typically includes an antibody comprising a polypeptide sequence that binds a target, wherein the target-binding polypeptide sequence was obtained by a process that includes the selection of a single target binding polypeptide sequence from a plurality of polypeptide sequences. For example, the selection process can be the selection of a unique clone from a plurality of clones, such as a pool of hybridoma clones, phage clones, or recombinant DNA clones. It should be understood that a selected target binding sequence can be further altered, for example, to improve affinity for the target, to humanize the target binding sequence, to improve its production in cell culture, to reduce its immunogenicity in vivo, to create a multispecific antibody, etc., and that an antibody comprising the altered target binding sequence is also a monoclonal antibody for purposes of this disclosure. In contrast to polyclonal antibody preparations, which typically include different antibodies directed against different determinants (epitopes), each monoclonal antibody of a monoclonal antibody preparation is directed against a single determinant on an antigen. In addition to their specificity, monoclonal antibody preparations are advantageous in that they are typically uncontaminated by other immunoglobulins.

The modifier term “monoclonal” indicates the character of the antibody as being obtained from a substantially homogeneous population of antibodies, and is not to be construed as requiring production of the antibody by any particular method. For example, the monoclonal antibodies to be used in accordance with the disclosure may be made by a variety of techniques, including, for example, the hybridoma method (e.g., Kohler and Milstein, Nature, 256:495-97 (1975); Hongo et al., Hybridoma, 14 (3): 253-260 (1995), Harlow et al., Antibodies: A Laboratory Manual, (Cold Spring Harbor Laboratory Press, 2nd ed. 1988); Hammerling et al., in: Monoclonal Antibodies and T-Cell Hybridomas 563-681 (Elsevier, N.Y., 1981)), recombinant DNA methods (see, e.g., U.S. Pat. No. 4,816,567), phage-display technologies (see, e.g., Clackson et al., Nature, 352:624-628 (1991); Marks et al., J. Mol. Biol. 222:581-597 (1992); Sidhu et al., J. Mol. Biol. 338 (2): 299-310 (2004); Lee et al., J. Mol. Biol. 340 (5): 1073-1093 (2004); Fellouse, Proc. Natl. Acad. Sci. USA 101 (34): 12467-12472 (2004); and Lee et al., J. Immunol. Methods 284 (1-2): 119-132 (2004), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Natl. Acad. Sci. USA 90:2551 (1993); Jakobovits et al., Nature 362:255-258 (1993); Bruggemann et al., Year in Immunol. 7:33 (1993); U.S. Pat. Nos. 5,545,807; 5,545,806; 5,569,825; 5,625,126; 5,633,425; and 5,661,016; Marks et al., Bio/Technology 10:779-783 (1992); Lonberg et al., Nature 368:856-859 (1994); Morrison, Nature 368:812-813 (1994); Fishwild et al., Nature Biotechnol. 14:845-851 (1996); Neuberger, Nature Biotechnol. 14:826 (1996); and Lonberg and Huszar, Intern. Rev. Immunol. 13:65-93 (1995).

A monoclonal antibody herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see, e.g., U.S. Pat. No. 4,816,567; and Morrison et al., Proc. Natl. Acad. Sci. USA 81:6851-6855 (1984)). Chimeric antibodies include antibodies wherein the antigen-binding region of the antibody is derived from an antibody produced by, e.g., immunizing macaque monkeys with the antigen of interest.

“Oligonucleotide,” as used herein, refers to short, typically single stranded polynucleotides that are generally, but not necessarily, less than about 200 nucleotides in length. The terms “oligonucleotide” and “polynucleotide” are not mutually exclusive. The description herein for polynucleotides is equally and fully applicable to oligonucleotides.

The term “operably linked” refers to functional linkage or association between a first component and a second component such that each component can be functional. For example, operably linked includes the association between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. In the context of two polypeptides that are operably linked a first polypeptide functions in the manner it would independent of any linkage and the second polypeptide functions as it would absent a linkage between the two.

“Percent identity” in the context of two or more nucleic acids or polypeptide sequences, refers to two or more sequences that share a degree of similarity. Two sequences are “substantially identical” if two sequences have a specified percentage of amino acid residues or nucleotides that are the same (e.g., 60% identity, optionally 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity over a specified region, or, when not specified, over the entire sequence), when compared and aligned for maximum correspondence over a comparison window, or designated region as measured using one of the following sequence comparison algorithms or by manual alignment and visual inspection. Optionally, the identity exists over a region that is at least about 30 nucleotides (or 10 amino acids) in length, or more preferably over a region that is 100 to 500 or 1000 or more nucleotides (or 20, 50, 200 or more amino acids) in length.

For sequence comparison, generally one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. Methods of alignment of sequences for comparison are well known in the art. Optimal alignment of sequences for comparison can be conducted, e.g., by the local homology algorithm of Smith and Waterman, (1970) Adv. Appl. Math. 2:482c, by the homology alignment algorithm of Needleman and Wunsch, (1970) J. Mol. Biol. 48:443, by the search for similarity method of Pearson and Lipman, (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection (see, e.g., Brent et al., (2003) Current Protocols in Molecular Biology).

Two examples of algorithms that can be used for determining percent sequence identity and sequence similarity are the BLAST and BLAST 2.0 algorithms, which are described in Altschul et al., (1977) Nuc. Acids Res. 25:3389-3402; and Altschul et al., (1990) J. Mol. Biol. 215:403-410, respectively. Software for performing BLAST analyses is publicly available through the National Center for Biotechnology Information.

The percent identity between two amino acid sequences can also be determined using the algorithm of E. Meyers and W. Miller, (1988) Comput. Appl. Biosci. 4:11-17) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (1970) J. Mol. Biol. 48:444-453) algorithm which has been incorporated into the GAP program in the GCG software package (available at [www.]gcg.com), using either a Blossom 62 matrix or a PAM250 matrix, and a gap weight of 16, 14, 12, 10, 8, 6, or 4 and a length weight of 1, 2, 3, 4, 5, or 6.

A “polynucleotide,” or “nucleic acid,” as used herein, refer to polymers of nucleotides of any length, and include DNA and RNA. The nucleotides can be deoxyribonucleotides, ribonucleotides, modified nucleotides or bases, and/or their analogs that can be incorporated into a polymer by DNA or RNA polymerase, or by a synthetic reaction. A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after synthesis, such as by conjugation with a label. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid or semi-solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, alpha-anomeric sugars, epimeric sugars such as arabinose, xyloses or lyxoses, pyranose sugars, furanose sugars, sedoheptuloses, acyclic analogs and a basic nucleoside analogs such as methyl riboside. One or more phosphodiester linkages may be replaced by alternative linking groups. These alternative linking groups include, but are not limited to, embodiments wherein phosphate is replaced by P(O)S (“thioate”), P(S)S (“dithioate”), “(O)NR₂ (“amidate”), P(O)R, P(O)OR′, CO or CH₂ (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

A “protein” or “polypeptide”, which terms are used interchangeably herein, comprises one or more chains of chemical building blocks called amino acids that are linked together by chemical bonds called peptide bonds.

“Single-chain Fv” or “scFv” refers to a fusion protein comprising at least one antibody fragment comprising a variable region of a light chain and at least one antibody fragment comprising a variable region of a heavy chain, wherein the light and heavy chain variable regions are contiguously linked, e.g., via a synthetic linker, e.g., a short flexible polypeptide linker, and capable of being expressed as a single chain polypeptide, and wherein the scFv retains the specificity of the intact antibody from which it is derived. Unless specified, as used herein an scFv may have the vL and vH variable regions in either order, e.g., with respect to the N-terminal and C-terminal ends of the polypeptide, the scFv may comprise vL-linker-vH or may comprise vH-linker-vL.

The term “substantially similar” or “substantially the same,” as used herein, denotes a sufficiently high degree of similarity between two numeric values (for example, one associated with an antibody of the disclosure and the other associated with a reference/comparator antibody), such that one of skill in the art would consider the difference between the two values to be of little or no biological and/or statistical significance within the context of the biological characteristic measured by the values (e.g., K_{d} values). The difference between said two values is, for example, less than about 50%, less than about 40%, less than about 30%, less than about 20%, and/or less than about 10% as a function of the reference/comparator value.

The phrase “substantially reduced,” “substantially increased,” or “substantially different,” as used herein, denotes a sufficiently high degree of difference between two numeric values (generally one associated with a molecule and the other associated with a reference/comparator molecule) such that one of skill in the art would consider the difference between the two values to be of statistical significance within the context of the biological characteristic measured by said values (e.g., K_{d} values). The difference between said two values is, for example, greater than about 10%, greater than about 20%, greater than about 30%, greater than about 40%, and/or greater than about 50% as a function of the value for the reference/comparator molecule.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called complementarity-determining regions or hypervariable regions (CDRs or HVRs, used interchangeably herein) both in the light-chain and the heavy-chain variable domains. The more highly conserved portions of variable domains are called the framework regions (FR). The variable domains of native heavy and light chains each comprise four FRs, largely adopting a 3-sheet configuration, connected by three HVRs, which form loops connecting, and in some cases forming part of, the 3-sheet structure. The HVRs in each chain are held together in close proximity by the FRs and, with the HVRs from the other chain, contribute to the formation of the antigen-binding site of antibodies (see Kabat et al., Sequences of Proteins of Immunological Interest, Fifth Edition, National Institute of Health, Bethesda, Md. (1991)). The constant domains are not involved directly in binding an antibody to an antigen, but exhibit various effector functions, such as participation of the antibody in antibody-dependent cellular toxicity.

A “variant Fc region” comprises an amino acid sequence which differs from that of a native sequence Fc region by virtue of at least one amino acid modification, typically one or more amino acid substitution(s). Typically, the variant Fc region has at least one amino acid substitution compared to a native sequence Fc region or to the Fc region of a parent polypeptide, e.g. from about one to about ten amino acid substitutions, and typically from about one to about five amino acid substitutions in a native sequence Fc region or in the Fc region of the parent polypeptide. The variant Fc region of a disclosure possesses at least about 80% homology with a native sequence Fc region and/or with an Fc region of a parent polypeptide, at least about 90% homology therewith, and typically at least about 95% homology therewith.

The term “vector,” as used herein, is intended to refer to a nucleic acid molecule capable of transporting another nucleic acid to which it has been linked. One type of vector is a “plasmid”, which refers to a circular double stranded DNA loop into which additional DNA segments may be ligated. Another type of vector is a phage vector. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell and replicate along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids.

Calcific aortic valve stenosis (CAVS) is the common form of acquired valvular heart disease, present in 3% of the population over 75 years of age. While risk factors are similar for CAVS and atherosclerosis, ~50% or more of patients with CAVS do not have clinically significant coronary artery disease, suggesting related but unique pathophysiology. Although surgical aortic valve replacement (SAVR) remains the gold standard treatment for most patients, at least one third of symptomatic patients with CAVS may not undergo SAVR. To fill this clinical need, transcatheter aortic valve replacement (TAVR) is increasingly being used, but overall survival remains modest due to the advanced age and other co-morbidities. With the aging of the population, the prevalence of CAVS will increase rapidly and portends medical, financial and ethical burdens to healthcare systems worldwide. Hence, identification of causal pathways mediating CAVS can provide novel targets for earlier therapy prior to end-stage disease. One of these pathways involves lipoprotein-associated phospholipase A₂ (Lp-PLA₂) and lipoprotein(a) (Lp(a)) axis.

The disclosure provides antibodies, antibody fragments and humanized antibodies that bind to Lp(a) in an isoform independent selectivity. Antibody fragments may be generated by traditional means, such as enzymatic digestion, or by recombinant techniques. In certain circumstances there are advantages of using antibody fragments, rather than whole antibodies. The smaller size of the fragments allows for rapid clearance, and may lead to improved access to tumors, plaques and diseased tissue. For a review of certain antibody fragments, see Hudson et al. (2003) Nat. Med. 9:129-134, herein incorporated by reference in its entirety.

Various techniques have been developed for the production of antibody fragments. Traditionally, these fragments were derived via proteolytic digestion of intact antibodies (see, e.g., Morimoto et al., Journal of Biochemical and Biophysical Methods 24:107-117 (1992); and Brennan et al., Science, 229:81 (1985)). However, these fragments can now be produced directly by recombinant host cells. Fab, Fv and ScFv antibody fragments can all be expressed in and secreted from E. coli, thus allowing the facile production of large amounts of these fragments. Antibody fragments can be isolated from the antibody phage libraries. Alternatively, Fab′-SH fragments can be directly recovered from E. coli and chemically coupled to form F(ab′)₂ fragments (Carter et al., Bio/Technology 10:163-167 (1992), herein incorporated by reference in its entirety). According to another approach, F(ab′)₂ fragments are isolated directly from recombinant host cell culture. Fab and F(ab′)₂ fragment with increased in vivo half-life comprising salvage receptor binding epitope residues are described in U.S. Pat. No. 5,869,046, herein incorporated by reference in its entirety. Other techniques for the production of antibody fragments will be apparent to the skilled practitioner. In certain embodiments, an antibody is a single chain Fv fragment (scFv). See WO 93/16185; U.S. Pat. Nos. 5,571,894; and 5,587,458, each of which is herein incorporated by reference in its entirety. Fv and scFv are the only species with intact combining sites that are devoid of constant regions; thus, they may be suitable for reduced nonspecific binding during in vivo use. scFv fusion proteins may be constructed to yield fusion of an effector protein at either the amino or the carboxy terminus of an scFv. See Antibody Engineering, ed. Borrebaeck, supra. The antibody fragment may also be a “linear antibody”, e.g., as described in U.S. Pat. No. 5,641,870, for example. Such linear antibodies may be monospecific or bispecific.

In addition, knowing the sequences of the antibody and antibody fragments, the generation of chimeric antigen receptors (CARs) and derivative (e.g., 1^{st} generation, 2^{nd} generation, 3^{rd} generation CARs) can be developed. Methods of cloning and generating CARs are known in the art. For example, the disclosure provides nucleic acid and polypeptide sequences of monoclonal antibodies and the respective CDRs/HVRs of the antibody. One of skill in the art can readily clone the binding domain to transmembrane domain (without or without linkers) of a chimeric antigen receptor to generate a CAR with selectivity to Lp(a).

The disclosure also provides for single chain variable antibody fragments (“scFv”), V_{H}, V_{L} and complementarity determining regions that selectively bind to lipoprotein(a) in an isoform independent manner. The scFvs of the disclosure are soluble and can be readily synthesized. Further, vectors comprising sequences encoding the scFvs disclosed herein enabled the production of a transgenic murine model for further studies of various diseases and research.

The disclosure, although providing specific antibody sequences and antibody sequence fragments having biological activity, is not limited to polypeptides having these specific sequences. In some embodiments, variants having a percent sequence identity with the specific antibody sequences of the disclosure are also provided. In some embodiments, such variants may be selected for use based on one or more desired, enhanced properties. It should be appreciated that certain variants may have an improved function associated with one desired feature (e.g., binding affinity, avoidance of undesired side effects, etc.), but have a less desirable function associated with another desired featured (e.g., stability, specificity, etc.).

In some embodiments, amino acid sequence modification(s) of the antibodies and fragments described herein are contemplated. For example, it may be desirable to improve the binding affinity and/or other biological properties of the antibody or fragment. Amino acid sequence variants of the antibody may be prepared by introducing appropriate changes into the nucleotide sequence encoding the antibody, or by peptide synthesis. Such modifications include, for example, deletions from, and/or insertions into and/or substitutions of, residues within the amino acid sequences of the antibody or antibody fragment. Any combination of deletion, insertion, and substitution can be made to arrive at the final construct, provided that the final construct possesses the desired characteristics comprising, at least binding to Lp(a). The amino acid alterations may be introduced in the subject antibody amino acid sequence at the time that sequence is made.

A useful method for identification of certain residues or regions of the antibody that are preferred locations for mutagenesis is called “alanine scanning mutagenesis” as described by Cunningham and Wells (1989) Science, 244:1081-1085, herein incorporated by reference in its entirety. Here, a residue or group of target residues are identified (e.g., charged residues such as Arg, Asp, His, Lys, and Glu) and replaced by a neutral or negatively charged amino acid (e.g., Alanine or Polyalanine) to affect the interaction of the amino acids with antigen. Those amino acid locations demonstrating functional sensitivity to the substitutions then are refined by introducing further or other variants at, or for, the sites of substitution. Thus, while the site for introducing an amino acid sequence variation is predetermined, the nature of the mutation per se need not be predetermined. For example, to analyze the performance of a mutation at a given site, Ala scanning or random mutagenesis is conducted at the target codon or region and the expressed immunoglobulins are screened for the desired activity.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g. for ADEPT) or a polypeptide which increases the serum half-life of the antibody. Polyhistidine tags are also useful for purification.

In certain embodiments, an antibody or antibody fragment of the disclosure is altered to increase or decrease the extent to which the antibody is glycosylated. Glycosylation of polypeptides is typically either N-linked or O-linked. N-linked refers to the attachment of a carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used.

Addition or deletion of glycosylation sites to the antibody is conveniently accomplished by altering the amino acid sequence such that one or more of the above-described tripeptide sequences (for N-linked glycosylation sites) is created or removed. The alteration may also be made by the addition, deletion, or substitution of one or more serine or threonine residues to the sequence of the original antibody (for O-linked glycosylation sites).

Where the antibody comprises an Fc region, the carbohydrate attached thereto may be altered. Native antibodies produced by mammalian cells typically comprise a branched, biantennary oligosaccharide that is generally attached by an N-linkage to Asn of the CH₂ domain of the Fc region. See, e.g., Wright et al. (1997) TIBTECH 15:26-32, herein incorporated by reference in its entirety. The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody of the invention may be made in order to create antibody variants with certain improved properties.

For example, antibody variants are provided having a carbohydrate structure that lacks fucose attached (directly or indirectly) to an Fc region. Such variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87:614 (2004), each of which is herein incorporated by reference in its entirety. Examples of cell lines capable of producing defucosylated antibodies include Lec13 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87:614 (2004); Kanda, Y. et al., Biotechnol. Bioeng., 94 (4): 680-688 (2006); and WO2003/085107), each of which is herein incorporated by reference in its entirety.

Antibody variants are further provided with bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.), each of which is herein incorporated by reference in its entirety. Antibody variants with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.); and WO 1999/22764 (Raju, S.), each of which is herein incorporated by reference in its entirety.

In certain embodiments, an antibody variant comprises an Fc region with one or more amino acid substitutions which further improve or inhibit/reduce ADCC. Such substitutions may occur in combination with any of the variations described above.

In certain embodiments, the disclosure contemplates an antibody variant that possesses some but not all effector functions, which make it a desirable candidate for many applications in which the half-life of the antibody in vivo is important yet certain effector functions (such as complement and ADCC) are unnecessary or deleterious. In certain embodiments, the Fc activities of the antibody are measured to ensure that only the desired properties are maintained. In vitro and/or in vivo cytotoxicity assays can be conducted to confirm the reduction/depletion of CDC and/or ADCC activities. For example, Fc receptor (FcR) binding assays can be conducted to ensure that the antibody lacks a particular binding but retains other binding. Non-limiting examples of in vitro assays to assess ADCC activity of a molecule of interest is described in U.S. Pat. No. 5,500,362 (see, e.g. Hellstrom, I., et al. Proc. Nat'l Acad. Sci. USA 83:7059-7063 (1986)) and Hellstrom, I et al., Proc. Nat'l Acad. Sci. USA 82:1499-1502 (1985); U.S. Pat. No. 5,821,337 (see Bruggemann, M. et al., J. Exp. Med. 166:1351-1361 (1987)), each of which is herein incorporated by reference in its entirety. Alternatively, non-radioactive assays methods may be employed. Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998), herein incorporated by reference in its entirety. C1q binding assays may also be carried out to confirm that the antibody is unable to bind C1q and hence lacks CDC activity. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)), each of which is herein incorporated by reference in its entirety. FcRn binding and in vivo clearance/half-life determinations can also be performed using methods known in the art (see, for example, Petkova, S. B. et al., Int'l. Immunol. 18 (12): 1759-1769 (2006) herein incorporated by reference in its entirety).

Other antibody variants having one or more amino acid substitutions are provided. Sites of interest for substitutional mutagenesis include the hypervariable regions, but FR alterations are also contemplated. Conservative substitutions can also be performed. Amino acid substitutions may be introduced into an antibody of interest and the products screened, e.g., for a desired activity, such as improved antigen binding, decreased immunogenicity, improved ADCC or CDC, etc.

The disclosure provides an antibody or antibody fragment capable of binding to Lp(a) in an isoform independent manner, wherein the antibody or antibody fragment comprises a variable heavy chain (V_{H}) domain and/or a variable light chain (V_{L}) domain, and wherein (a) the V_{H} domain comprises an amino acid sequence that includes one, two and three complementarity determining regions (CDRs) including, but not limited to:
(i) SEQ ID NO:9 or variants thereof;
(ii) SEQ ID NO:10 or variants thereof; and
(iii) SEQ ID NO:11 or variants thereof; and
   (b) the V_{L} domain comprises an amino acid sequence that includes one, two or three complementarity determining regions (CDRs) including, but not limited to:
(i) SEQ ID NO:12 or variants thereof;
(ii) SEQ ID NO:13 or variants thereof; and
(iii) SEQ ID NO:14 or variants thereof.

In one embodiment, the antibody or antibody fragment comprises a V_{H} domain that comprises an amino acid sequence that includes CDRs comprising SEQ ID NO:9, 10 and 11, and/or the V_{L} domain comprises an amino acid sequence that includes CDRs comprising SEQ ID NO:12, 13 and 14.

In one embodiment, the disclosure provides an antibody or an scFv selected from the group consisting of an antibody or scFv with heavy and light chain domains comprising the complementarity determining regions of SEQ ID NO:9, 10, 11, 12, 13, and 14. In one embodiment the scFv is linked to an Fc region.

In one embodiment, the disclosure provides an antibody comprising a light-chain variable region as set forth in SEQ ID NO:16 lacking the signal peptide. In another embodiment, the disclosure provides an antibody that comprises a heavy chain variable region comprising a sequence of SEQ ID NO:18 lacking the signal peptide.

In one embodiment, the disclosure provides an scFv comprising a linker between the light change variable region and the heavy-chain variable region. The linker can be any number of commonly used peptide linkers. In one embodiment, the linker comprises one or more repeating units of GGGS (SEQ ID NO:19). The repeat of GGGS (SEQ ID NO:19) may be 2, 3, 4 or more times.

In another embodiment, the disclosure comprises a scFv comprising a light chain variable region of SEQ ID NO:16 linked by a peptide linker to a heavy chain variable region of SEQ ID NO:18. In another embodiment, the disclosure provides for an scFv having a light chain that has a polypeptide sequence that has at least 75%, at least 80%, at least 85%, at least 90%, at least 95%, at least 98%, or at least 99% sequence identity to SEQ ID NO:16, and a heavy chain that has a polypeptide sequence that has at least 75%, at least 80%, at least 85%, at least 90%, at least 95%, at least 98%, or at least 99% sequence identity to SEQ ID NO:18, contains the CDRs of SEQ ID NO:9, 10, 11, 12, 13, and 14, and wherein the scFv selectively binds to a lipoprotein(a).

In yet further embodiments, fusion constructs comprising a first domain comprising SEQ ID NO:16 and/or 18 or a variant or fragment thereof is operably linked to a second domain comprising (i) a detectable label or (ii) a polypeptide of interest. One of skill in the art will recognize that such fusion constructs can be generated using chemical or molecular biology techniques that link a coding sequence comprising a sequence of SEQ ID NO:15 and/or 17 or variants thereof with a coding sequence of, for example, a polypeptide of interest. The coding sequences and domains may be separated by a linker or directly linked.

In yet another embodiment, the disclosure comprises a scFv comprising a light chain variable region of SEQ ID NO:16 linked by a peptide linker to a heavy chain variable region of SEQ ID NO:18.

Nucleic acid molecules encoding the amino acid sequences of the antibodies, antibody fragments and variants of the antibody are prepared by a variety of methods known in the art. For preparing variants such methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of the antibody.

In a particular embodiment, the disclosure provides a polynucleotide encoding a heavy chain of an antibody or antibody fragment of the disclosure comprising (i) a sequence that encodes a polypeptide of SEQ ID NO:18; (ii) a sequence comprising SEQ ID NO:17 or (iii) a sequence that is at least 80% identical and/or hybridizes to a sequence consisting of SEQ ID NO:17.

In a particular embodiment, the disclosure provides a polynucleotide encoding a light chain of an antibody or antibody fragment of the disclosure comprising (i) a sequence that encodes a polypeptide of SEQ ID NO:16; (ii) a sequence comprising SEQ ID NO:15 or (iii) a sequence that is at least 80% identical and/or hybridizes to a sequence consisting of SEQ ID NO:15.

The disclosure further provides for a scFv disclosed herein that further comprises a fragment crystallizable region (“Fc”) of an antibody. In a particular embodiment, the Fc region is from a human or humanized antibody and may be modified to modulate the domains biological activity. The Fc region is the tail region of an antibody that interacts with cell surface receptors called Fc receptors and some proteins of the complement system. This property allows antibodies to activate the immune system. In IgG, IgA and IgD antibody isotypes, the Fc region is composed of two identical protein fragments, derived from the second and third constant domains of the antibody's two heavy chains; IgM and IgE Fc regions contain three heavy chain constant domains (CH domains 2-4) in each polypeptide chain. The Fc regions of IgGs bear a highly conserved N-glycosylation site. Glycosylation of the Fc fragment is essential for Fc receptor-mediated activity. The N-glycans attached to this site are predominantly core-fucosylated biantennary structures of the complex type. In addition, small amounts of these N-glycans also bear bisecting GlcNAc and α-2,6 linked sialic acid residues. The other part of an antibody, called the Fab region, contains variable sections that define the specific target that the antibody can bind. The scFv of the disclosure are comprised of elements from the Fab region. By contrast, the Fc region of all antibodies in a class are the same for each species; they are constant rather than variable. The Fc region is, therefore, sometimes termed the “fragment constant region”. Accordingly, the polynucleotide and polypeptide sequences which encode the Fc regions for countless species have already been determined and would be known by one of skill in the art. In a particular embodiment, the disclosure provides for an antibody and antibody fragment polynucleotides encoding SEQ ID NO:16 and/or 18 or a variant thereof that further comprises a polynucleotide sequence which encodes an Fc region.

In a particular embodiment, the disclosure provides for a polynucleotide sequence disclosed herein (e.g., SEQ ID NO:15 and/or 17) that further comprises a polynucleotide sequence which encodes an Fc region.

In a further embodiment, the disclosure provides for a vector which comprises a polynucleotide sequence encoding an antibody or antibody fragment wherein the polynucleotide comprises SEQ ID NO:15 and/or 17 or sequences having sequence identity of at least 99%, at least 95%, at least 90%, at least 85%, at least 80%, at least 75% or at least 70% identity to SEQ ID NO:15 and/or 17 and which encodes a polypeptide that specifically binds to a lipoprotein(a). In a further embodiment, the disclosure provides for a vector which comprises a polynucleotide sequence encoding an antibody or antibody fragment wherein the polynucleotide comprises SEQ ID NO:15 and/or 17 or sequences having sequence identity of at least 99%, at least 95%, at least 90%, at least 85%, at least 80%, at least 75% or at least 70% identity to SEQ ID NO:15 and/or 17 and which contains the CDRs of SEQ ID NO:9, 10, 11, 12, 13, and 14.

Polynucleotide sequences encoding polypeptide components of the antibody or antibody fragments of the disclosure can be obtained using standard recombinant techniques. Desired polynucleotide sequences may be isolated and sequenced from antibody producing cells such as hybridoma cells.

Alternatively, polynucleotides can be synthesized using nucleotide synthesizer or PCR techniques. Once obtained, sequences encoding the polypeptides are inserted into a recombinant vector capable of replicating and expressing heterologous polynucleotides in prokaryotic hosts. Many vectors that are available and known in the art can be used for the purpose of the disclosure. Selection of an appropriate vector will depend mainly on the size of the nucleic acids to be inserted into the vector and the particular host cell to be transformed with the vector. Each vector contains various components, depending on its function (amplification or expression of heterologous polynucleotide, or both) and its compatibility with the particular host cell in which it resides. The vector components generally include, but are not limited to: an origin of replication, a selection marker gene, a promoter, a ribosome binding site (RBS), a signal sequence, the heterologous nucleic acid insert and a transcription termination sequence.

In general, plasmid vectors containing replicon(s) and control sequences which are derived from species compatible with the host cell are used in connection with these hosts. The vector ordinarily carries a replication site, as well as marking sequences which are capable of providing phenotypic selection in transformed cells. For example, E. coli is typically transformed using pBR322, a plasmid derived from an E. coli species. pBR322 contains genes encoding ampicillin (Amp) and tetracycline (Tet) resistance and thus provides easy means for identifying transformed cells. pBR322, its derivatives, or other microbial plasmids or bacteriophage may also contain, or be modified to contain, promoters which can be used by the microbial organism for expression of endogenous proteins. Examples of pBR322 derivatives used for expression of particular antibodies are described in detail in Carter et al., U.S. Pat. No. 5,648,237.

In addition, phage vectors containing replicon and control sequences that are compatible with the host microorganism can be used as transforming vectors in connection with these hosts. For example, bacteriophage vectors may be utilized in making a recombinant vector which can be used to transform susceptible host cells such as E. coli LE392.

The expression vector of the disclosure may comprise two or more promoter-cistron pairs, encoding each of the polypeptide components. A promoter is an untranslated regulatory sequence located upstream (5′) to a cistron that modulates its expression. Prokaryotic promoters typically fall into two classes, inducible and constitutive. Inducible promoter is a promoter that initiates increased levels of transcription of the cistron under its control in response to changes in the culture condition, e.g. the presence or absence of a nutrient or a change in temperature.

A large number of promoters recognized by a variety of potential host cells are well known. The selected promoter can be operably linked to cistron DNA encoding the light or heavy chain by removing the promoter from the source DNA via restriction enzyme digestion and inserting the isolated promoter sequence into the vector of the invention. Both the native promoter sequence and many heterologous promoters may be used to direct amplification and/or expression of the target genes. In some embodiments, heterologous promoters are utilized, as they generally permit greater transcription and higher yields of expressed target gene as compared to the native target polypeptide promoter.

Promoters suitable for use with prokaryotic hosts include the PhoA promoter, the (3-galactanase and lactose promoter systems, a tryptophan (trp) promoter system and hybrid promoters such as the tac or the trc promoter. However, other promoters that are functional in bacteria (such as other known bacterial or phage promoters) are suitable as well. Their nucleotide sequences have been published, thereby enabling a skilled worker operably to ligate them to cistrons encoding the target light and heavy chains (Siebenlist et al. (1980) Cell 20:269) using linkers or adaptors to supply any required restriction sites.

In one embodiment, each cistron within the recombinant vector comprises a secretion signal sequence component that directs translocation of the expressed polypeptides across a membrane. In general, the signal sequence may be a component of the vector, or it may be a part of the target polypeptide DNA that is inserted into the vector (See Table A and B and SEQ ID Nos:16 and 18). The signal sequence selected for the purpose of the disclosure should be one that is recognized and processed (e.g., cleaved by a signal peptidase) by the host cell. For prokaryotic host cells that do not recognize and process the signal sequences native to the heterologous polypeptides, the signal sequence is substituted by a prokaryotic signal sequence selected, for example, from the group consisting of the alkaline phosphatase, penicillinase, Ipp, or heat-stable enterotoxin II (STII) leaders, LamB, PhoE, PelB, OmpA and MBP. In one embodiment of the invention, the signal sequences used in both cistrons of the expression system are STII signal sequences or variants thereof.

In another embodiment, the production of the immunoglobulins according to the disclosure can occur in the cytoplasm of the host cell, and therefore does not require the presence of secretion signal sequences within each cistron. In that regard, immunoglobulin light and heavy chains are expressed, folded and assembled to form functional immunoglobulins within the cytoplasm. Certain host strains (e.g., the E. coli trxB-strains) provide cytoplasm conditions that are favorable for disulfide bond formation, thereby permitting proper folding and assembly of expressed protein subunits. Proba and Pluckthun Gene, 159:203 (1995).

Prokaryotic host cells suitable for expressing antibodies of the disclosure include Archaebacteria and Eubacteria, such as Gram-negative or Gram-positive organisms. Examples of useful bacteria include Escherichia (e.g., E. coli), Bacilli (e.g., B. subtilis), Enterobacteria, Pseudomonas species (e.g., P. aeruginosa), Salmonella typhimurium, Serratia marcescans, Klebsiella, Proteus, Shigella, Rhizobia, Vitreoscilla, or Paracoccus. In one embodiment, gram-negative cells are used. In one embodiment, E. coli cells are used as hosts for the disclosure. Examples of E. coli strains include strain W3110 (Bachmann, Cellular and Molecular Biology, vol. 2 (Washington, D.C.: American Society for Microbiology, 1987), pp. 1190-1219; ATCC Deposit No. 27, 325) and derivatives thereof, including strain 33D3 (U.S. Pat. No. 5,639,635). Other strains and derivatives thereof, such as E. coli 294 (ATCC 31, 446), E. coli B, E. coli X 1776 (ATCC 31, 537) and E. coli RV308 are also suitable. These examples are illustrative rather than limiting. Methods for constructing derivatives of any of the above-mentioned bacteria having defined genotypes are known in the art and described in, for example, Bass et al., Proteins, 8:309-314 (1990). It is generally important to select the appropriate bacteria taking into consideration replicability of the replicon in the cells of a bacterium. For example, E. coli, Serratia, or Salmonella species can be suitably used as the host when well-known plasmids such as pBR322, pBR325, pACYC177, or pKN410 are used to supply the replicon. Typically the host cell should secrete minimal amounts of proteolytic enzymes, and additional protease inhibitors may desirably be incorporated in the cell culture.

Host cells are transformed with the above-described expression vectors and cultured in conventional nutrient media modified as appropriate for inducing promoters, selecting transformants, or amplifying the genes encoding the desired sequences.

Transformation means introducing DNA into the prokaryotic host so that the DNA is replicable, either as an extrachromosomal element or by chromosomal integrant. Depending on the host cell used, transformation is done using standard techniques appropriate to such cells. The calcium treatment employing calcium chloride is generally used for bacterial cells that contain substantial cell-wall barriers. Another method for transformation employs polyethylene glycol/DMSO. Yet another technique used is electroporation.

Prokaryotic cells used to produce the polypeptides of the disclosure are grown in media known in the art and suitable for culture of the selected host cells. Examples of suitable media include luria broth (LB) plus necessary nutrient supplements. In some embodiments, the media also contains a selection agent, chosen based on the construction of the expression vector, to selectively permit growth of prokaryotic cells containing the expression vector. For example, ampicillin is added to media for growth of cells expressing ampicillin resistant gene.

Any necessary supplements besides carbon, nitrogen, and inorganic phosphate sources may also be included at appropriate concentrations introduced alone or as a mixture with another supplement or medium such as a complex nitrogen source. Optionally the culture medium may contain one or more reducing agents selected from the group consisting of glutathione, cysteine, cystamine, thioglycollate, dithioerythritol and dithiothreitol. The prokaryotic host cells are cultured at suitable temperatures.

In one embodiment, the expressed polypeptides are secreted into and recovered from the periplasm of the host cells. Protein recovery typically involves disrupting the microorganism, generally by such means as osmotic shock, sonication or lysis. Once cells are disrupted, cell debris or whole cells may be removed by centrifugation or filtration. The proteins may be further purified, for example, by affinity resin chromatography. Alternatively, proteins can be transported into the culture media and isolated therein. Cells may be removed from the culture and the culture supernatant being filtered and concentrated for further purification of the proteins produced. The expressed polypeptides can be further isolated and identified using commonly known methods such as polyacrylamide gel electrophoresis (PAGE) and Western blot assay.

Large scale or small scale fermentation can be used and can be optimized using skills well known in the art.

Standard protein purification methods known in the art can be employed. The following procedures are exemplary of suitable purification procedures: fractionation on immunoaffinity or ion-exchange columns, ethanol precipitation, reverse phase HPLC, chromatography on silica or on a cation-exchange resin such as DEAE, chromatofocusing, SDS-PAGE, ammonium sulfate precipitation, and gel filtration.

The disclosure further provides for an expression vector which can be replicated in a prokaryotic system or a eukaryotic system, the vector comprising at least one polynucleotide encoding a light and/or heavy chain disclosed herein. In one embodiment, the disclosure provides for an expression vector which comprises a polynucleotide sequence encoding a first sequence having identity of at least 100%, at least 99%, at least 95%, at least 90%, at least 85%, at least 80%, at least 75% or at least 70% identity to SEQ ID NO:16. In another embodiment, a second vector or the same vector comprises a second polynucleotide encoding a second sequence having identity of at least 100%, at least 99%, at least 95%, at least 90%, at least 85%, at least 80%, at least 75% or at least 70% identity to SEQ ID NO:18. In a further embodiment, the vector, when expressed, results in an scFv or antibody in a soluble form, and possesses the ability to bind to Lp(a) in an isoform independent manner.

The disclosure further provides for an expression vector which encodes a scFv or antibody disclosed herein that is transferred into a suitable host organism. The suitable host organism is a microorganism, yeast or a mammalian cell system. Typically, the mammalian cell system is monocyte-derived (e.g., macrophages, monocytes, and neutrophils), lymphocyte-derived (e.g., myeloma, hybridoma, and a normal immortalized B cell), parenchymal (e.g., hepatocytes) and non-parenchymal cells (e.g., stellate cells).

The scFv disclosed herein which further comprises an Fc region, can be used for many possible therapeutic uses. For example, they can be used as an anti-atherosclerotic agent.

In one embodiment, of the disclosure a method for determining a subject's Lp(a) amounts and/or predisposition to coronary artery disease is provided. The method includes determining a subject's plasma Lp(a) and correlating the Lp(a) level control or normal levels, wherein increased levels of Lp(a) are indicative of a predisposition to coronary artery disease. The amount of Lp(a) can be detected using an antibody, antibody fragment or binding domain comprising the CDRs for the light and heavy chains as described herein that specifically binds to an Lp(a) in an isoform independent manner. The disclosure can further use a second antibody that binds to OxPL such as EO6, MB47, LPA4. Or a secondary antibody that binds to another epitope of Lp(a) (e.g., KIV9; see, WO 2021/222181, the disclosure of which is incorporated herein by reference in its entirety).

The method can be carried out on a biological sample obtained from a subject. The biological sample can be, for example, blood, serum, or plasma.

In some embodiments, the antibody, antibody fragment or binding domain are immobilized on a substrate to form an array. In one embodiment, the substrate is a microtiter plate, a glass slide, a nylon slide, a nitrocellulose membrane or a PVDF membrane. In another embodiment, the substrate is a latex bead, a glass bead or a microsphere.

An exemplary biochemical test for identifying Lp(a), employs a standardized test format, such as the Enzyme Linked Immunosorbent Assay or ELISA test, although the information provided herein may apply to the development of other biochemical or diagnostic tests and is not limited to the development of an ELISA test. Various commercially available ELISA kits are available. Other assay formats can be adapted to use the antibody or antibody fragments of the disclosure, such assay formats including latex beads and the like.

In one embodiment, an immunoassay can be performed either by first capturing the Lp(a) on a microtiter well by use of an antibody of the disclosure, and then detection of the Lp(a) by a labeled antibody. In another embodiment, a bead-based assay can be utilized with fluorescently barcoded beads comprising an antibody or antibody-fragment linked to the bead, wherein the assays is interrogated using flow cytometry methods. Bead-based immunoassays enable multiplex detection of targets several folds higher than what is conceivable using normal ELISA formats, saving time and sample.

The disclosure also provides that a scFv or an antibody of the disclosure can be used as an anti-atherosclerotic agent to treat cardiovascular disease and CAVS. A scFv or antibody of the disclosure can be used to treat high risk patients with coronary artery disease (“CAD”), by inhibiting clot formation induced by the prothrombotic activity of Lp(a).

The scFvs and antibodies disclosed herein can bind to Lp(a) s in an isoform independent manner and block the formation of plaques, clot and/or atherogenesis. It is anticipated that the in vivo use of an scFv, antibody or antibody fragment of the disclosure can be used to block Lp(a) biological effects in many different situations.

As used herein, “treatment” refers to clinical intervention in an attempt to alter the natural course of the individual or cell being treated, and can be performed either for prophylaxis or during the course of clinical pathology. Desirable effects of treatment include preventing occurrence or recurrence of disease, alleviation of symptoms, diminishment of any direct or indirect pathological consequences of the disease, decreasing the rate of disease progression, amelioration or palliation of the disease state, and remission or improved prognosis. In some embodiments, a scFv, antibody or antibody fragment of the disclosure is used to delay development of a disease or disorder.

An “individual,” “subject,” or “patient” is a vertebrate. In certain embodiments, the vertebrate is a mammal. Mammals include, but are not limited to, farm animals (such as bovine, equine, sus), sport animals, pets/companion animals (such as Felis catus, canine), primates, mice and rats. In certain embodiments, a mammal is a human.

An “effective amount” refers to an amount effective, at dosages and for periods of time necessary, to achieve the desired therapeutic or prophylactic result.

A “therapeutically effective amount” of a substance/molecule of the disclosure, agonist or antagonist may vary according to factors such as the disease state, age, sex, and weight of the individual, and the ability of the substance/molecule, agonist or antagonist to elicit a desired response in the individual. A therapeutically effective amount is also one in which any toxic or detrimental effects of the substance/molecule, agonist or antagonist are outweighed by the therapeutically beneficial effects. A “prophylactically effective amount” refers to an amount effective, at dosages and for periods of time necessary, to achieve the desired prophylactic result. Typically, but not necessarily, since a prophylactic dose is used in subjects prior to or at an earlier stage of disease, the prophylactically effective amount will be less than the therapeutically effective amount.

Therapeutic formulations comprising an antibody or fragment thereof of the disclosure are prepared for storage by mixing the antibody or fragment having the desired degree of purity with optional physiologically acceptable carriers, excipients or stabilizers (Remington: The Science and Practice of Pharmacy 20th edition (2000)), in the form of aqueous solutions, lyophilized or other dried formulations. Acceptable carriers, excipients, or stabilizers are nontoxic to recipients at the dosages and concentrations employed, and include buffers such as phosphate, citrate, histidine and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants or polyethylene glycol (PEG).

The formulation herein may also contain more than one active compound as necessary for the particular indication being treated, preferably those with complementary activities that do not adversely affect each other. Such molecules are suitably present in combination in amounts that are effective for the purpose intended.

The active ingredients may also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Remington: The Science and Practice of Pharmacy 20th edition (2000).

The formulations to be used for in vivo administration must be sterile. This is readily accomplished by filtration through sterile filtration membranes.

Sustained-release preparations may be prepared. Suitable examples of sustained-release preparations include semipermeable matrices of solid hydrophobic polymers containing the immunoglobulin of the disclosure, which matrices are in the form of shaped articles, e.g., films, or microcapsule. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γethyl-L-glutamate, non-degradable ethylene-vinyl acetate, degradable lactic acid-glycolic acid copolymers such as the LUPRON DEPOT (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated immunoglobulins remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

Anti-Lp(a) antibodies of the disclosure can be produced transgenically through the generation of a mammal or plant that is transgenic for the immunoglobulin heavy and light chain sequences of interest and production of the antibody in a recoverable form therefrom. In connection with the transgenic production in mammals, anti-Lp(a) antibodies can be produced in, and recovered from, the milk of goats, cows, or other mammals. See, e.g., U.S. Pat. Nos. 5,827,690, 5,756,687, 5,750,172, and 5,741,957, incorporated herein by reference.

In some embodiments, non-human transgenic animals or plants are produced by introducing one or more nucleic acid molecules encoding an anti-Lp(a) antibody or fragment thereof (e.g., SEQ ID NO:15 and/or 17) of the disclosure into the animal or plant by standard transgenic techniques. See Hogan and U.S. Pat. No. 6,417,429. The transgenic cells used for making the transgenic animal can be embryonic stem cells or somatic cells or a fertilized egg. The transgenic non-human organisms can be chimeric, nonchimeric heterozygotes, and nonchimeric homozygotes. See, e.g., Hogan et al., Manipulating the Mouse Embryo: A Laboratory Manual 2nd ed., Cold Spring Harbor Press (1999); Jackson et al., Mouse Genetics and Transgenics: A Practical Approach, Oxford University Press (2000); and Pinkert, Transgenic Animal Technology: A Laboratory Handbook, Academic Press (1999), all incorporated herein by reference. In some embodiments, the transgenic non-human animals have a targeted disruption and replacement by a targeting construct that encodes a heavy chain and/or a light chain of interest. In another embodiment, the transgenic animals comprise and express nucleic acid molecules encoding heavy and light chains that specifically bind to epitopes on Lp(a). In some embodiments, the transgenic animals comprise nucleic acid molecules encoding a modified antibody such as a single-chain antibody, a chimeric antibody or a humanized antibody. The anti-Lp(a) antibodies may be made in any transgenic animal. In another embodiment, the non-human animals are mice, rats, sheep, pigs, goats, cattle or horses. The non-human transgenic animal expresses said encoded polypeptides in blood, milk, urine, saliva, tears, mucus and other bodily fluids.

The following examples are intended to illustrate but not limit the disclosure. While they are typical of those that might be used, other procedures known to those skilled in the art may alternatively be used.

### EXAMPLES

The following peptides were generated:

pKIV4
(SEQ ID NO: 2)
CSDAEWTAFVPPNVILAPSLEAFFEQ

Peptides were resuspended in sterile PBS (1 mg/ml). The resuspended peptides were used as antigens for immunization.

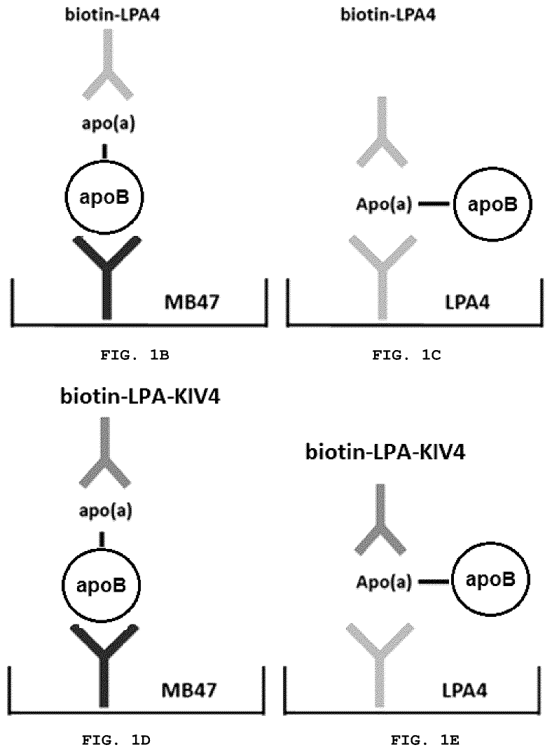

BALB/C female mice (n=2) were injected SQ with 250 ug of antigen (50 ug of 5 peptides pooled) in 250 ul incomplete Freund's adjuvant on day 0, 14 and 28. IgG titers against K8 were tested in immunized mice plasma on day 38 (FIG. 2) followed by a 4^{th} SQ boost (250 ug/250 ul in IcFA) on day 42.

Three days post I.V. boost (125 ug peptide mix in PBS) splenocytes from mouse #2 fused with myeloma cells (p3X63Ag8.653) using ClonaCell™-HY Hybridoma kit (StemCell Technologies, Cambridge MA), and the fused cells were resuspended in semi-solid HAT hybridoma selection medium.

After 10 days, ~300 colonies were transferred to ClonaCell®-HY Growth Medium (DMEM, pre-selected serum, HT, gentamycin and supplements).

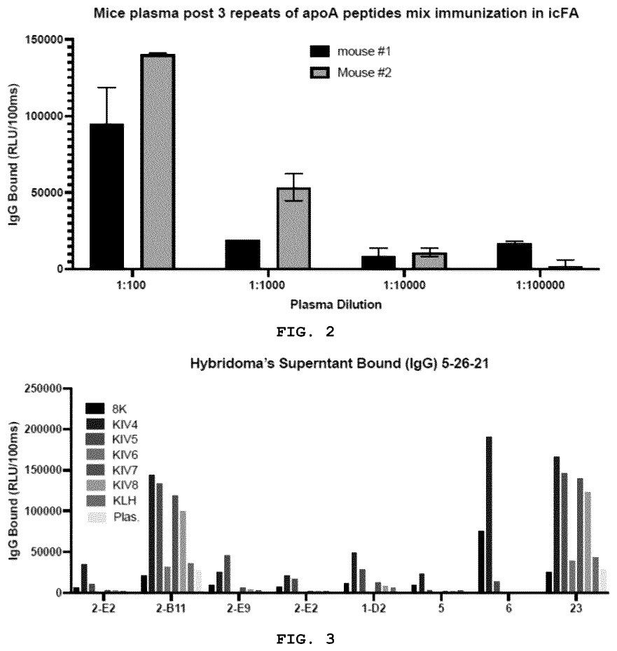

To select colonies expressing antibodies specific to apo(a) without binding to plasminogen or KIV2, conditioned media from the hybridoma cells were screened by ELISA against an array of antigens (FIG. 3).

TABLE A
LPA-KIV4 Heavy chain (polynucleotide sequence = SEQ ID NO: 17;
polypeptide sequence = SEQ ID NO: 18; CDR1=SEQ ID NO: 9;
CDR2 = SEQ ID NO: 10; CDR3 = SEQ ID NO:11)

```
Heavy chain: DNA sequence (399 bp)
Signal sequence-FR1-CDR1-FR2-CDR2-FR3-CDR3-FR4
ATGGAATGTAACTGGATACTTCCTTTTATTCTGTCGGTAACTTCAGGGGTCTACTCAGAGGCTCAGC
                              Signal Sequence
TCCAGCAGTCTGGGACTGTGCTGGCAAGGCCTGGGGCTTCAGTGAAGATGTCCTGCAAGGCTTCTGG
CTACACCTTTACCAACTACTGGATGCACTGGGTAAAACAGGGGCCTGGACAGGGTCTGGAATGGATT
                                  CDR1
GGCGCTATTTATCCTGGAAATAGTGATACTTACTACAACCAGAAGTTCAAGGGCAAGGCCAAACTGA
                                            CDR2
CTGCAGTCACATCCACCACCACTGCCTACATGGAGCTCAGCAGCCTGACAAATGAGGACTCTGCGGT
CTATTACTGTTCGAACTGGGACTTAGACTACTGGGGCCAAGGCACCACTCTCACAGTCTCCTCA
                                          CDR3
Heavy chain: Amino acid sequence (133 aa)
Signal peptide-FR1-CDR1-FR2-CDR2-FR3-CDR3-FR4
MECNWILPFILSVTSGVYSEAQLQQSGTVLARPGASVKMSCKASGYTFNYWMHWVKQGPGQGLEWI
        Signal Sequence CDR1
GAIYPGNSDTYYNQKFKGKAKLTAVTSTTTAYMELSSLTNEDSAVYYCSNWDLDYWGQGTTLTVSS
          CDR2 CDR3
```

TABLE B
LPA-KIV4 Light chain (polynucleotide sequence = SEQ ID NO : 15;
polypeptide sequence = SEQ ID NO: 16; CDR1=SEQ ID NO: 12; CDR2 =
SEQ ID NO: 13; CDR3 = SEQ ID NO: 14)

```
Light chain: DNA sequence (393 bp)
Signal sequence-FR1-CDR1-FR2-CDR2-FR3-CDR3-FR4
ATGAGTCCTGCCCAGTTCCTGTTTCTGTTAGTGCTCTGGATTCAGGAAACCAACGGTGATGTTGTGA
                                      Signal Sequence
TGACCCAGACTCCACTCACTTTGTCGGTTACCATTGGCCAACCAGCCTCTATCTCTTGCAAGTCAAG
TCAGAGCCTCTTATATAGTAATGGAAAAACCTTTTTGAATTGGTTATTGCACAGGCCAGGCCAGTCT
                        CDR1
CCAAAGCGCCTAATCTATCTGGTGTCTAAACTGGACTCTGGAGTCCCTGACAGGTTCACTGGCAGTG
                                              CDR2
GATCAGGAATAGATTTTACACTGAAAATCAGCAGAGTGGAGGCTGAGGATTTGGGAGTTTATTACTG
CGTGCAAGGTACACATTTTCCGTACACGTTCGGAGGGGGGACCAAGCTGGAAATAAAA
                    CDR3
Light chain: Amino acid sequence (131 aa)
Signal peptide-FR1-CDR1-FR2-CDR2-FR3-CDR3-FR4
MSPAQFLFLLVLWIQETNGUVVMTQTPLTLSVTIGQPASISCKSSQSLLYSNGKTFLNWLLHRPGQS
      Signal Sequence CDR1
PKRLIYLVSKLDSGVPDRFTGSGSGIDHTLKIGRVEAEDLGVYYCVQGTHFPYTFGGGTKLEIK
              CDR2 CDR3
```

A number of embodiments have been described herein. Nevertheless, it will be understood that various modifications may be made without departing from the spirit and scope of this disclosure. Accordingly, other embodiments are within the scope of the following claims.

1. An antibody or antibody fragment that recognizes and binds to lipoprotein(a), wherein the antibody or antibody fragment comprises a variable heavy chain (V_{H}) domain and/or a variable light chain (V_{L}) domain, and wherein (a) the V_{H} domain comprises an amino acid sequence that includes complementarity determining regions (CDRs) of: SEQ ID NO:9 or variants thereof; SEQ ID NO:10 or variants thereof; and SEQ ID NO:11 or variants thereof; and (b) the V_{L} domain comprises an amino acid sequence that includes complementarity determining regions (CDRs) of: SEQ ID NO:12 or variants thereof; SEQ ID NO:13 or variants thereof; and SEQ ID NO:14 or variants thereof,
wherein the antibody or antibody fragment binds to kringle IV type 4 (KIV4) of lipoprotein(a).

2. The antibody or antibody fragment of claim 1, wherein the V_{H} domain comprises an amino acid sequence of SEQ ID NO:18, and the V_{L} domain comprises an amino acid sequence of SEQ ID NO:16.

3. The antibody or an antibody fragment of claim 1, wherein the antibody or antibody fragment is selected from the group consisting of an antibody or scFv with heavy and light chain domains comprising the complementarity determining regions of SEQ ID NO:9, 10, 11, 12, 13, and 14.

4. The antibody or antibody fragment of claim 1, wherein the heavy and light chain domains are linked to an Fc region.

5. An antibody fragment of claim 1 comprising a single chain variable fragment (“scFv”) that recognizes an epitope of KIV4 of lipoprotein(a).

6. (canceled)

7. The scFv of claim 5, wherein the scFv comprises a light-chain variable region having a sequence that is at least 95% identical to the sequence as set forth in SEQ ID NO:16 and binds to KIV4.

8. The scFv of claim 7, wherein the scFv comprises a heavy chain variable region having a sequence that is at least 95% identical to the sequence as set forth in SEQ ID NO:18 and binds to KIV4.

9. An antibody comprising a variable light chain and variable heavy chain of claim 1.

10. (canceled)

11. A pharmaceutical composition comprising an antibody of claim 9 and a carrier, excipient, or stabilizer.

12. An antibody or antibody fragment of claim 1 bound to a solid substrate.

13. An antibody or antibody fragment of claim 1 operably linked to a detectable label.

14. A polynucleotide that encodes an antibody, antibody fragment, variable light chain, variable heavy chain or scFv of claim 1.

15. The polynucleotide of claim 14, wherein the polynucleotide (i) comprises a sequence that hybridizes to a nucleic acid consisting of SEQ ID NO:15 and/or 17 and encodes an antibody that binds to KIV4 of Lp(a); and (ii) comprises a sequence that encodes a polypeptide having a sequence of SEQ ID NO:16 and/or 18.

16. A vector comprising a polynucleotide of claim 15.

17. A host cell transformed with the polynucleotide of claim 15.

18. A host cell transformed with a vector of claim 16.

19-22. (canceled)

23. A method of treating or preventing coronary artery disease or calcific aortic stenosis comprising administering an antibody or antibody fragment of claim 1 to a subject having or at risk of having coronary artery disease.

24. A chimeric antigen receptor (CAR) comprising a binding domain containing the VH and/or VL domains of claim 1.

25. A CAR-T cell comprising the chimeric antigen receptor (CAR) of claim 24.

26. An isolated peptide sequence consisting of SEQ ID NO:1 having 1-10 amino acids linked at the N-terminal or C-terminal ends.

27. An immunological composition comprising a peptide of SEQ ID 1 and an adjuvant.

28. A pharmaceutical compositions comprising an isolated peptide of claim 26.

29. An antigenic epitope that is specifically recognized by a monoclonal antibody, wherein the monoclonal antibody comprises the complementarity determining regions of SEQ ID NO:9, 10, 11, 12, 13, and 14.

30. (canceled)

31. An immunoassay comprising an antibody or antibody fragment of claim 1.

32. The immunoassay of claim 31, wherein the antibody or antibody fragment is linked to a solid substrate.


SEQUENCE LISTING (19 sequences)

SEQ ID NO: 1 - protein, 7 aa, synthetic construct
LVPPNVI

SEQ ID NO: 2 - protein, 26 aa, synthetic construct
CSDAEWTAFVPPNVILAPSLEAFFEQ

SEQ ID NO: 3 - protein, 26 aa, synthetic construct
CLVTESSVLATLTVVPDPSTEASSEE

SEQ ID NO: 4 - protein, 18 aa, synthetic construct
CPVTESSVLATSTAVSEQ

SEQ ID NO: 5 - protein, 26 aa, synthetic construct
CPVMESTLLTTPTVVPVPSTELPSEE

SEQ ID NO: 6 - protein, 26 aa, synthetic construct
CPVTESSVLTTPTVAPVPSTEAPSEQ

SEQ ID NO: 7 - protein, 7 aa, synthetic construct
LETPTVV

SEQ ID NO: 8 - protein, 23 aa, synthetic construct
CSETESGVLETPTVVPVPSMEAH

SEQ ID NO: 9 - protein, 5 aa, synthetic construct
NYWMH

SEQ ID NO: 10 - protein, 17 aa, synthetic construct
AIYPGNSDTYYNQKFKG

SEQ ID NO: 11 - protein, 5 aa, synthetic construct
WDLDY

SEQ ID NO: 12 - protein, 16 aa, synthetic construct
KSSQSLLYSNGKTFLN

SEQ ID NO: 13 - protein, 7 aa, synthetic construct
LVSKLDS

SEQ ID NO: 14 - protein, 9 aa, synthetic construct
VQGTHFPYT

SEQ ID NO: 15 - DNA, 393 bp, synthetic construct
  source 1..393: mol_type = other DNA
atgagtcctgcccagttcctgtttctgttagtgctctggattcaggaaaccaacggtgatgttgtgatgacccagactccactcactttgtcggttaccattggccaaccagcctctatctcttgcaagtcaagtcagagcctcttatatagtaatggaaaaacctttttgaattggttattgcacaggccaggccagtctccaaagcgcctaatctatctggtgtctaaactggactctggagtccctgacaggttcactggcagtggatcaggaatagattttacactgaaaatcagcagagtggaggctgaggatttgggagtttattactgcgtgcaaggtacacattttccgtacacgttcggaggggggaccaagctggaaataaaa

SEQ ID NO: 16 - protein, 131 aa, synthetic construct
MSPAQFLFLLVLWIQETNGDVVMTQTPLTLSVTIGQPASISCKSSQSLLYSNGKTFLNWLLHRPGQSPKRLIYLVSKLDSGVPDRFTGSGSGIDFTLKISRVEAEDLGVYYCVQGTHFPYTFGGGTKLEIK

SEQ ID NO: 17 - DNA, 399 bp, synthetic construct
  source 1..399: mol_type = other DNA
atggaatgtaactggatacttccttttattctgtcggtaacttcaggggtctactcagaggctcagctccagcagtctgggactgtgctggcaaggcctggggcttcagtgaagatgtcctgcaaggcttctggctacacctttaccaactactggatgcactgggtaaaacaggggcctggacagggtctggaatggattggcgctatttatcctggaaatagtgatacttactacaaccagaagttcaagggcaaggccaaactgactgcagtcacatccaccaccactgcctacatggagctcagcagcctgacaaatgaggactctgcggtctattactgttcgaactgggacttagactactggggccaaggcaccactctcacagtctcctca

SEQ ID NO: 18 - protein, 133 aa, synthetic construct
MECNWILPFILSVTSGVYSEAQLQQSGTVLARPGASVKMSCKASGYTFTNYWMHWVKQGPGQGLEWIGAIYPGNSDTYYNQKFKGKAKLTAVTSTTTAYMELSSLTNEDSAVYYCSNWDLDYWGQGTTLTVSS

SEQ ID NO: 19 - protein, 4 aa, synthetic construct
GGGS